(19) United States
(12) Patent Application Publication
(10) Pub. No.: US 2021/0290564 A1
(43) Pub. Date: Sep. 23, 2021

(54) MEDICATED CANNABINOID COMPOSITIONS, METHODS OF MANUFACTURING, AND METHODS OF TREATMENT

(71) Applicant: Tauriga Sciences Inc., Wappingers Falls, NY (US)

(72) Inventor: Seth Marcus Shaw, New York, NY (US)

(21) Appl. No.: 17/204,106

(22) Filed: Mar. 17, 2021

Related U.S. Application Data
(60) Provisional application No. 62/990,709, filed on Mar. 17, 2020.

(51) Int. Cl. A61K 31/05; A61K 9/68; A61K 31/12

(52) U.S. Cl. CPC A61K 31/05; A61K 31/12; A61K 9/0058

(57) ABSTRACT

Disclosed in certain embodiments is a composition for treating nausea and/or vomiting. The composition could be chewable or any other composition suitable for oral administration. The composition may include at least one cannabinoid components, such as cannabidiol and/or cannabigerol, and a ginger component(s). The composition may include any one of these components or any combination of these components in an effective amount to treat nausea and/or vomiting. Also disclosed herein are methods of preparing a composition to treat nausea and/or vomiting and methods of treating nausea and/or vomiting.

### RELATED APPLICATIONS

This application claims priority to U.S. Provisional Patent Application No. 62/990,709, filed on Mar. 17, 2020, which is herein incorporated by reference in its entirety.

### FIELD OF THE INVENTION

In certain embodiments, the present invention relates to the field of pharmaceutical compositions for treating nausea and/or vomiting, methods of preparation thereof, and methods for treating nausea and/or vomiting.

### BACKGROUND OF THE INVENTION

Cancer treatments, such as chemotherapy and radiation therapy, can cause nausea and vomiting. Some drugs, such as targeted therapy and immunotherapy, can also cause nausea and vomiting. Some types of cancers may also contribute to nausea and vomiting. Nausea and/or vomiting may also be triggered due to peripheral factors (such as substance intoxication), due to psychological factors (such as anxiety), under post-operative circumstances, due to pregnancy, due to motion sickness, and the like.

Individuals are often provided medicines to prevent nausea and vomiting from starting and/or to manage and/or treat the nausea and vomiting when it begins. Various types of anti-emetic drugs are currently available for a variety of types of nausea and vomiting. Some examples of such drugs include, serotonin (5-HT3) antagonists for acute nausea, NK-1 receptor antagonists for delayed nausea, steroids, dopamine antagonists, and olanzapine, to name a few.

Cannabis contains many chemical compounds useful for medicinal or recreational applications due to their high concentration of cannabinoids. Cannabinoids may also assist with preventing and/or treating nausea and/or vomiting experienced by oncology subjects. Cannabinoids may also assist with preventing and/or treating nausea and/or vomiting triggered by various other reasons.

Cannabis is a complex plant with over 400 chemical entities of which more than 60 of them are cannabinoid compounds, some of them with opposing effects. [See: Cannabis, a complex plant: different compounds and different effects on subjects. Zerrin Atakan, Ther Adv Psychopharmacol. 2012 December; 2 (6): 241-254. doi: 10.1177/2045125312457586 and references therein; Downloaded Mar. 8, 2021 from https://www.ncbi.nlm.nih.gov/pmc/articles/PMC3736954/]

The cannabinoid family of compounds include such compounds as delta-9-THC, delta-8-THC, cannabinol, cannbidiol, delta-9-tetrahydrocannabivarin, cannabigerol and cannabichromene.

There exists a need in the art for a pharmaceutical composition for treating nausea and/or vomiting in a subject, a method for preparing said pharmaceutical composition, and a method for treating nausea and/or vomiting in a subject in need thereof.

### OBJECTS AND SUMMARY OF THE INVENTION

It is an object of certain embodiments of the present invention to provide a pharmaceutical composition (e.g., an oral composition such as a chewable composition, a thin film, a spray, a suspension, or the like) for preventing nausea in a subject in need thereof (e.g., a cancer subject, a person experiencing motion sickness, a person experiencing substance intoxication, pregnant individuals, a person that has undergone an operation and experiences post-operative nausea and/or vomiting symptoms, or the like).

It is an object of certain embodiments of the present invention to provide a pharmaceutical composition (e.g., an oral composition such as a chewable composition, a thin film, a spray, a suspension, or the like) for treating nausea in a subject in need thereof (e.g., a cancer subject, a person experiencing motion sickness, a person experiencing substance intoxication, pregnant individuals, a person that has undergone an operation and experiences post-operative nausea and/or vomiting symptoms, or the like).

It is an object of certain embodiments of the present invention to provide a pharmaceutical composition (e.g., an oral composition such as a chewable composition, a thin film, a spray, a suspension, or the like) for preventing vomiting in a subject in need thereof (e.g., a cancer subject, a person experiencing motion sickness, a person experiencing substance intoxication, pregnant individuals, a person that has undergone an operation and experiences post-operative nausea and/or vomiting symptoms, or the like).

It is an object of certain embodiments of the present invention to provide a pharmaceutical composition (e.g., an oral composition such as a chewable composition, a thin film, a spray, a suspension, or the like) for treating vomiting in a subject in need thereof (e.g., a cancer subject, a person experiencing motion sickness, a person experiencing substance intoxication, pregnant individuals, a person that has undergone an operation and experiences post-operative nausea and/or vomiting symptoms, or the like).

It is an object of certain embodiments of the present invention to provide a method for treating nausea in a subject in need thereof (e.g., a cancer subject, a person experiencing motion sickness, a person experiencing substance intoxication, pregnant individuals, a person that has undergone an operation and experiences post-operative nausea and/or vomiting symptoms, or the like).

It is an object of certain embodiments of the present invention to provide a method for treating vomiting in a subject in need thereof (e.g., a cancer subject, a person experiencing motion sickness, a person experiencing substance intoxication, pregnant individuals, a person that has undergone an operation and experiences post-operative nausea and/or vomiting symptoms, or the like).

It is an object of certain embodiments of the present invention to provide a method for preventing nausea in a subject in need thereof (e.g., a cancer subject, a person experiencing motion sickness, a person experiencing substance intoxication, pregnant individuals, a person that has undergone an operation and experiences post-operative nausea and/or vomiting symptoms, or the like).

It is an object of certain embodiments of the present invention to provide a method for preventing vomiting in a subject in need thereof (e.g., a cancer subject, a person experiencing motion sickness, a person experiencing substance intoxication, pregnant individuals, a person that has undergone an operation and experiences post-operative nausea and/or vomiting symptoms, or the like).

It is an object of certain embodiments of the present invention to provide a method for manufacturing a pharmaceutical composition suitable for treatment and/or prevention of nausea and/or vomiting in a subject in need thereof (e.g., a cancer subject, a person experiencing motion sickness, a person experiencing substance intoxication, pregnant individuals, a person that has undergone an operation and experiences post-operative nausea and/or vomiting symptoms, or the like).

The above objects and others may be achieved by the present invention which in certain embodiments is directed to a pharmaceutical composition (e.g., an oral composition such as a chewable composition, a thin film, a spray, a suspension, or the like) for providing nausea and/or vomiting treatment to a subject in need thereof, or for preventing nausea and/or vomiting in a subject in need thereof, wherein the pharmaceutical composition includes one or more cannabinoid components in an effective amount to treat nausea and/or vomiting and a ginger component(s). In certain embodiments, the pharmaceutical composition includes one or more cannabinoid components in an effective amount to treat nausea and/or vomiting, a ginger component(s), and a chewable base.

In certain embodiments, the pharmaceutical compositions described herein refer to an oral composition that includes one or more cannabinoid components and a ginger component(s), wherein the one or more cannabinoid components and the ginger component(s), together, are present in the oral composition in an effective amount to treat and/or prevent nausea and/or vomiting in a subject in need thereof.

In certain embodiments, the pharmaceutical compositions described herein refer to a chewable composition that includes one or more cannabinoid components, a ginger component(s), and a chewable base, wherein the one or more cannabinoid components and the ginger component(s), together, are present in the chewable composition in an effective amount to treat and/or prevent nausea and/or vomiting in a subject in need thereof.

In certain embodiments, the present disclosure is directed to a method for treating and/or preventing nausea and/or vomiting in a subject in need thereof. In one embodiments, the method includes administering to a subject in need thereof any of the chewable compositions described herein. For instance, in one embodiment, the chewable composition includes one or more cannabinoid components and a ginger component(s). In another embodiment, the method includes administering the pharmaceutical composition to the subject by applying or placing the composition in the oral cavity of the subject. In certain embodiments, the composition may be left in the oral cavity of the subject undisturbed for a duration sufficient for the pharmaceutical composition to disintegrate and/or dissolve.

In certain embodiments, particular subjects that may benefit from the method described herein are cancer patients that could experience nausea and/or vomiting due to any number of reasons, such as, without limitations, the cancer treatment regime, the cancer itself, side effects to other drugs, and so on. Other subjects who could experience nausea and/or vomiting due to any number of reasons, such as, underlying medical conditions, as a post-operative symptom, substance intoxication, motion sickness, pregnancy, psychological factors, and the like, may also benefit from the pharmaceutical compositions described herein.

In certain embodiments, the present disclosure is directed to a method of manufacturing a pharmaceutical composition (e.g., an oral composition such as a chewable composition, a thin film, a spray, a suspension, or the like) that is suitable for treating and/or preventing nausea and/or vomiting in a subject in need thereof. In certain embodiments, the method includes combining the one or more cannabinoid components with the ginger component(s) and a chewable base to form any of the chewable compositions described herein. In other embodiments, the method may include formulating one or more cannabinoid components with a ginger component(s) into any of the oral dosage forms described herein (tablets, capsules, caplets, films, sprays, drops, liquid formulations, suspension, emulsions, powders, sublingual tablets, and the like).

In certain embodiments, the present disclosure is directed to a kit that includes a container and any of the pharmaceutical compositions described herein stored within the container. The container may be a bottle, a bag, a blister package, or any other suitable packaging for any of the pharmaceutical compositions described herein. In certain embodiments, the container may be a packaging suitable for a chewable composition. In certain embodiments, the container may include a delivery device, such as, a spray delivery device, an aerosol delivery device, a muco-adhesive delivery system, and the like. In certain embodiments, the pharmaceutical compositions may be packaged in single dose containers (e.g., a blow seal unit dose).

In certain embodiments, the one or more cannabinoid components includes, without limitations, cannabidiol, cannabigerol, tetrahydrocannabinol, cannabinol, cannbichromene, or a combination thereof. In one embodiment, the chewable composition includes a combination of cannabidiol and cannabigerol.

In certain embodiments, the ginger component(s) includes, without limitations, ginger, a ginger root extract, a shogaol, a zingerone, a gingerol, or a combination thereof. In one embodiment, the chewable composition includes gingerol as the ginger component(s).

In certain embodiments, the cannabidiol is present in any of the oral pharmaceutical compositions described herein in a therapeutically effective amount to treat and/or prevent nausea and/or vomiting in a subject in need thereof. In certain embodiments, the cannabigerol is present in any of the oral pharmaceutical compositions described herein in a therapeutically effective amount to treat and/or prevent nausea and/or vomiting in a subject in need thereof. In certain embodiments, the gingerol is present in any of the oral pharmaceutical compositions described herein in a therapeutically effective amount to treat and/or prevent nausea and/or vomiting in a subject in need thereof. In certain embodiments, two or more of the cannabidiol, cannabigerol, and gingerol are, together, present in any of the oral pharmaceutical compositions described herein in a therapeutically effective amount to treat and/or prevent nausea and/or vomiting in a subject in need thereof.

In certain embodiments, the cannabidiol is present in the chewable composition in a therapeutically effective amount to treat and/or prevent nausea and/or vomiting in a subject in need thereof. In certain embodiments, the cannabigerol is present in the chewable composition in a therapeutically effective amount to treat and/or prevent nausea and/or vomiting in a subject in need thereof. In certain embodiments, the gingerol is present in the chewable composition in a therapeutically effective amount to treat and/or prevent nausea and/or vomiting in a subject in need thereof. In certain embodiments, two or more of the cannabidiol, cannabigerol, and gingerol are, together, present in the chewable composition in a therapeutically effective amount to treat and/or prevent nausea and/or vomiting in a subject in need thereof.

### Definitions

As used herein, the singular forms “a,” “an,” and “the” include plural references unless the context clearly indicates otherwise. Thus, for example, reference to “an antioxidant” includes a single antioxidant as well as a mixture of two or more different antioxidants; reference to “a ginger component” includes a single ginger component as well as a mixture of two or more different ginger components; reference to “an excipient” includes a single excipient as well as a mixture of two or more different excipients; reference to “a cannabinoid component” includes a single cannabinoid component as well as a mixture of two or more different cannabinoid components; and the like.

As used herein, the term “about” in connection with a measured quantity, refers to the normal variations in that measured quantity, as expected by one of ordinary skill in the art in making the measurement and exercising a level of care commensurate with the objective of measurement. In certain embodiments, the term “about” includes the recited number ±10%, such that “about 10” would include from 9 to 11.

As used herein, the term “active agent” or “active ingredient” refers to any material that is intended to produce a therapeutic, prophylactic, or other intended effect, whether or not approved by a government agency for that purpose. This term with respect to a specific agent includes the pharmaceutically active agent, and all pharmaceutically acceptable salts, solvates and crystalline forms thereof, where the salts, solvates and crystalline forms are pharmaceutically active. Pharmaceutically acceptable salts include, but are not limited to, inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate, and the like; amino acid salts such as arginate, asparginate, glutamate and the like, and metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like.

The term “solvate” refers to an aggregate that comprises one or more molecules of active agent with one or more molecules of a solvent. The solvent may be water, in which case the solvate may be a hydrate. Alternatively, the solvent may be an organic solvent. In one embodiment, “solvate” refers to the active pharmaceutical ingredient in its state prior to dissolution. Alternatively, the solid particles of a suspended active agent may comprise a co-precipitated solvent.

As used herein, the phrase “pharmaceutically acceptable” means that which is useful in preparing a pharmaceutical composition that is generally safe, non-toxic, and is not biologically or otherwise undesirable and is acceptable for human pharmaceutical use.

As used herein, the terms “therapeutically effective” and an “effective amount” refer to that amount of an active agent or the rate at which it is administered needed to produce a desired therapeutic result, such as to treat and/or prevent a condition or symptoms of a condition in a subject.

The term “subject” refers to a human or animal, who has demonstrated a clinical manifestation of nausea and/or vomiting suggesting the need for a nausea and/or vomiting treatment, or who is at risk of experiencing nausea and/or vomiting. The term “subject” may also refer to a human or an animal, who has demonstrated a clinical manifestation of symptoms that are associated or may be associated with nausea and/or vomiting, or who is at a risk of experiencing such symptoms, suggesting the need for a nausea and/or vomiting treatment. For example, a cancer/oncology subject who experiences nausea and/or vomiting from their existing cancer treatment regime (e.g., cytotoxic chemotherapy and/or radiotherapy) or who is about to start a cancer treatment regime that could cause nausea and/or vomiting and the subject is treated prophylactically with any of the chewable compositions described herein. The subject in need thereof may include individuals that are experiencing (or may experience) nausea and/or vomiting due to underlying medical conditions, substance intoxication, motion sickness, psychological factors, post operatively, pregnancy, and the like. In certain embodiments, a subject in need thereof may be an individual who experiences nausea and/or vomiting related to peripheral factors such as ingestion of toxins (e.g., alcohol/drug intoxication), disturbance of the vestibular system, peritoneal inflammation, and bowel obstruction. Yet another example may be an individual who experiences nausea and/or vomiting related disorders of delayed gastric emptying as, for example, in diabetes and idiopathic gastroparesis. Yet another example may be an individual who experiences nausea and/or vomiting that may be triggered by anxiety, threatening situation, a situation that is regarded as distasteful by the subject, or hostility (such as a temper tantrum). In certain embodiments, subject in need thereof refers to an individual who experiences nausea and/or vomiting that is post-operative and/or which may be attributed to the anesthetic agents and/or the analgesic agents that are administered to the individual. In certain embodiments, subject refers to an individual who experiences nausea and/or vomiting related to motion sickness (e.g., sea-sickness, car-sickness, air-sickness). In certain embodiments, the term subject in need thereof may refer to an individual experiencing nausea and/or vomiting that is pregnancy related (e.g., a pregnant individual experiencing morning sickness).

The terms “treatment of” and “treating” include the administration of an active agent(s) with the intent to lessen the severity of a condition.

The terms “prevention of” and “preventing” include the avoidance of the onset of a condition by a prophylactic administration of the active agent.

The term “condition” or “conditions” may refer to those medical conditions commonly recognized as nausea, vomiting, or symptoms thereof, such as dizziness, faintness, dry mouth, diarrhea, fever, abdominal pain, decreased urination, or a combination thereof, which can be treated, mitigated or prevented by a timely administration to a subject of the pharmaceutical oral composition described herein (such as a chewable composition, a thin film, a spray, a suspension, or the like). In certain embodiments, the nausea or vomiting may be physiological and may be triggered by peripheral factors such as ingestion of toxins (e.g., alcohol/drug intoxication), disturbance of the vestibular system, peritoneal inflammation and bowel obstruction. It may also occur in certain medical disorders of delayed gastric emptying as, for example, in diabetes and idiopathic gastroparesis. In certain embodiments, the nausea or vomiting may be psychogenic and may be triggered by anxiety, threatening situation, a situation that is regarded as distasteful by a subject, hostility (such as a temper tantrum). In certain embodiments, the nausea and/or vomiting may be induced by cytotoxic chemotherapy and/or radiotherapy. In certain embodiments, the nausea and/or vomiting may be post-operative which may be attributed to the anesthetic agents and/or the analgesic agents that are administered to the subject. In certain embodiments, the nausea and/or vomiting may be related to motion sickness (e.g., sea-sickness, car sickness, air sickness). In certain embodiments, the nausea and/or vomiting may be pregnancy related (e.g., morning sickness).

As used herein, “oral delivery” or “oral administration” refers to a route of administration wherein the pharmaceutical dosage form is taken through the mouth. Oral administration is a part of enteral administration, which also includes buccal (dissolved inside the cheek), sublabial (dissolved under the lip), and sublingual administration (dissolved under the tongue). Enteral medications come in various forms, including: tablets to swallow, chew or dissolve in water or under the tongue; capsules and chewable capsules (with a coating that dissolves in the stomach or bowel to release the medication there); time-release or sustained-release tablets and capsules (which release the medication gradually); powders or granules; teas; drops; and liquid medications or syrups. As used herein “oral composition” or “oral pharmaceutical composition” refers to dosage forms or enteral medications suitable for oral administration.

As used herein, “capsule” refers to a solid pharmaceutical oral dosage form wherein the active (and inactive) ingredient is encapsulated. Encapsulation refers to a range of techniques used to enclose medicines in a relatively stable shell known as a capsule, allowing them to, for example, be taken orally. The two main types of capsules include hard-shelled capsules, which are typically made using gelatin and contain dry, powdered ingredients or miniature pellets made by, e.g. processes of extrusion or spheronisation. These are made in two halves: a lower-diameter “body” that is filled and then sealed using a higher-diameter “cape”. The second main type of capsules include soft-shelled capsules, primarily used for oils and for active ingredients that are dissolved or suspended in oil. Both of these classes of capsules are made from aqueous solutions of gelling agents like such as animal protein mainly gelatin; and plant polysaccharides or their derivatives like carrageenan and modified forms of starch and cellulose. Other ingredients can be added to the gelling agent solution like plasticizers such as glycerin and/or sorbitol to decrease the capsule's hardness, coloring agents, preservatives, disintegrants, lubricants and surface treatment.

As used herein, “tablet” refers to a pharmaceutical dosage form that includes a mixture of active substances and excipients, usually in powder form, pressed or compacted from a powder into a solid dose. The excipients can include diluents, binders or granulating agents, glidants (flow aids) and lubricants to ensure efficient tableting; disintegrants to promote tablet break-up in the digestive tract; sweeteners or flavors to enhance taste; and pigments to make the tablets visually attractive. A polymer coating is often applied to make the tablet smoother and easier to swallow, to control the release rate of the active ingredient, to make it more resistant to the environment (extending its shelf life), or to enhance the tablet's appearance.

As used herein, “orally dissolving tablet” or “ODT” or “orally disintegrating tablet” refers to pharmaceutical dosage form designed to be dissolved on the tongue rather than swallowed whole. The ODT serves as an alternative dosage form for patients who experience dysphagia (difficulty in swallowing) or for where compliance is a known issue and therefore an easier dosage form to take ensures that medication is taken. Common among all age groups, dysphagia is observed in about 35% of the general population, as well as up to 60% of the elderly institutionalized population and 18-22% of all patients in long-term care facilities. Additional reasons to use ODTs include the convenience of a tablet that can be taken without water as well as the inability of the patient to eat or drink (e.g., nausea and/or vomiting).

As used herein, “oral thin film,” “OTF,” “oral dissolving film,” “oral drug strip,” “oral thin film,” “thin film,” “orally dissolvable film strip,” or “oral strip,” or “orally disintegrating film” refers to a product used to administer active ingredients via absorption in the mouth (buccally or sublingually), the stomach (gastrically), and/or via the small intestines (enterically). The OTF is edible and pharmaceutically acceptable. A film is prepared typically using hydrophilic polymers that rapidly dissolves on the tongue, palatine tissue, or buccal cavity, delivering the active ingredient to the systemic circulation via dissolution when contact with liquid is made. The OTF (or more appropriately “thin film” or “TF”) can also be used to adhere to mucosal tissue (e.g., in the mouth), thereby locally delivering the active ingredient(s). The term “film” includes thin films and sheets, in any shape, including rectangular, square, or other desired shape. The films described herein may be any desired thickness and size such that it may be placed into the oral cavity of the user. For example, the films may have a relatively thin thickness of from about 0.1 to about 10 mils, or they may have a somewhat thicker thickness of from about 10 mils to about 30 mils. For some films, the thickness may be even larger, i.e., greater than about 30 mils. In addition, the term “film” includes single-layer compositions as well as multi-layer compositions, such as laminated films. The composition in its dried film form can effectively maintain a relatively uniform distribution of components through the application of controlled drying of the film. For example, the film can have no more than a 20%, 10%, 5%, or 1% variance of the active ingredient, per unit area of the film.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

All references to wt. % throughout the specifications and the claims refer to the weight of the component in reference to the weight of the entire subject composition and may also be designated as w/w, unless explicitly indicated otherwise.

### DETAILED DESCRIPTION

### Pharmaceutical Composition

In certain embodiments, the present disclosure is directed to a composition for treatment of nausea and/or vomiting in a subject in need thereof. A subject in need thereof may be a cancer patient that is experiencing nausea and/or to vomiting or a cancer patient that anticipates experiencing nausea and/or vomiting due to treatment, due to the side effects of the cancer, due to other medicines given for health problems that are not cancer related, bowel slowdown or blockage (obstruction), constipation, imbalance of minerals and salts (electrolytes) in the blood, infections, anxiety, other diseases or illness, or any other cause. The subject in need thereof may include individuals that are experiencing (or may experience) nausea and/or vomiting due to underlying medical conditions, substance intoxication, motion sickness, psychological factors, post operatively, pregnancy, and the like. In certain embodiments, a subject in need thereof may be an individual who experiences (or may experience) nausea and/or vomiting related to peripheral factors such as ingestion of toxins (e.g., alcohol/drug intoxication), disturbance of the vestibular system, peritoneal inflammation, bowel obstruction, or other underlying medical conditions. Yet another example may be an individual who experiences (or may experience) nausea and/or vomiting related disorders of delayed gastric emptying as, for example, in diabetes and idiopathic gastroparesis. Yet another example may be an individual who experiences (or may experience) nausea and/or vomiting that may be triggered by anxiety, threatening situation, a situation that is regarded as distasteful by the subject, or hostility (such as a temper tantrum). In certain embodiments, subject in need thereof refers to an individual who experiences (or may experience) nausea and/or vomiting that is post-operative and/or which may be attributed to the anesthetic agents and/or the analgesic agents that are administered to the individual. In certain embodiments, subject refers to an individual who experiences (or may experience) nausea and/or vomiting related to motion sickness (e.g., sea-sickness, car-sickness, air-sickness). In certain embodiments, the term subject in need thereof may refer to an individual experiencing nausea and/or vomiting that is pregnancy related (e.g., a pregnant individual experiencing morning sickness).

In certain embodiments, the pharmaceutical compositions described herein may be used for treating nausea and/or vomiting at the time that the subject is experiencing them or as prophylactic treatment against anticipatory nausea and/or vomiting. In certain embodiments, the pharmaceutical compositions described herein may be used for treating symptoms associated with nausea and/or vomiting.

### Oral Dosage Forms

In certain embodiments, the compositions described herein may be in any form that is suitable for oral administration. In certain embodiments, the oral dosage forms described herein may be formulated for oral ingestion, or via mucosal administration such as via the sublingual route, buccal route, gingival route, in the entirety of the oral cavity (entire mouth space), or a combination thereof. In certain embodiments, the compositions may be in a form of a tablet, a capsule, caplets, a lozenge, a troche, a chewable tablet (such as a gum), a syrup, a liquid solution or suspension, an emulsion, a buccal film, a sublingual film, an oral adhesive film, a powder, solid crystals, an orally-disintegrating tablet, a paste, an oral cream, an oral gel, an oral ointment, and so on.

In certain embodiments, the compositions described herein may be in a form of a tablet, which may be prepared, e.g., via compression, via granulation (e.g., wet granulation or dry granulation), via extrusion, via tableting, via compaction, or a combination thereof. In certain embodiments, the tablet may include one or more of the active agents described herein. In certain embodiments, the tablet may include a plurality of layers (such as a core and shell structure or a core and multi-layer shell structure). When more than one active agent is included in the tablet, the active agents may be dispersed homogenously in various parts of the tablet, in certain embodiments. In certain embodiments, the active agents may be separated in various parts of the tablet (e.g., one active agent may be in the core and another active agent may be in the shell). In certain embodiments, the tablet may be coated (e.g., with a compression shell, spray coated, dip coated, cosmetic coating). In certain embodiments, the tablet may be formulated to attain a target disintegration and/or dissolution profile. In certain embodiments, the tablet may include an enteric coating (e.g., to target delivery to a certain location within the gastrointestinal tract).

In certain embodiments, the compositions described herein may in a form of a capsule (e.g., a hard shell capsule or a softgel capsule). In certain embodiments, the capsules may include a shell composition enclosing a fill composition. In certain embodiments, the fill composition of the capsule may be liquid, solid (e.g., tablet, beads, powder, particles within a capsule, mini-tablets), semi-solid, or a combination thereof. In certain embodiments, the shell composition may be animal based (e.g., gelatin) or non-animal based. In certain embodiments, the softgel capsule may be coated (e.g., spray coated, dip coated, or include a shell composition with several layers prepared, e.g., via rotary die). In certain embodiments, the capsule may be sealed (e.g., sealed seamlessly). In certain embodiments, the active agents may be separated in various parts of the capsule (e.g., one active agent may be in the fill in a liquid form and a second active agent may be in the fill in a solid form). In certain embodiments, the active agents may be dispersed homogenously in various parts of the capsule. In certain embodiments, the capsule may be formulated to attain a target disintegration and/or dissolution profile. In certain embodiments, the capsule may be enteric (e.g., to target delivery to a certain location within the gastrointestinal tract).

In certain embodiments, the compositions described herein may be in a form of lozenge, a torche, a powder formulation, a sprinkle formulation for food or beverages (e.g., powder or solid crystals), a chewable gum or gummy, a chewable tablet, a sublingual tablet, a muco-adhesive delivery system (such as buccal film), a paste (e.g., a toothpaste), an oral cream, an oral gel, an oral ointment, drops (e.g., a viscous liquid), a syrup, suitable for gingival administration route (e.g., a paste), or a combination thereof.

In certain embodiments, the compositions described herein may be formulated for sublingual administration (e.g., viscous liquid, spray, sublingual tablet, thin films, powder).

In certain embodiments, viscous liquid drops/liquid/emulsion/suspension may be pre-filled into unit dose (e.g., blow-fill-seal single use units). In certain embodiments, the compositions described herein may in a form of a viscous liquid, which includes one or more cannabinoid components, a ginger component(s), and pharmaceutically acceptable excipients suitable for forming a viscous liquid, wherein the one or more cannabinoid components and the ginger component(s), individually or together, are present in the viscous liquid composition in an effective amount to treat, reduce, and/or prevent nausea and/or vomiting.

In certain embodiments, spray formulations may be filled into a suitable delivery device suitable for delivering a powder/suspension/dispersion/emulsion/liquid composition. In certain embodiments, the compositions described herein may in a form of a spray, the spray may be similar to those known in the art (e.g., spray such as found in a nicotine spray product on the market) which includes one or more cannabinoid components, a ginger component(s), and pharmaceutically acceptable excipients suitable for forming a spray, wherein the one or more cannabinoid components and the ginger component(s), individually or together, are present in the spray composition in an effective amount to treat, reduce, and/or prevent nausea and/or vomiting.

In certain embodiments, the compositions described herein may be formulated as sublingual tablets (e.g., hydrophilic formulation or hydrophobic formulation). In certain embodiments, the compositions described herein may in a form of a sublingual tablet, which includes one or more cannabinoid components, a ginger component(s), and pharmaceutically acceptable excipients suitable for forming a hydrophilic or a hydrophobic sublingual tablet, wherein the one or more cannabinoid components and the ginger component(s), individually or together, are present in the sublingual tablet composition in an effective amount to treat, reduce, and/or prevent nausea and/or vomiting.

In certain embodiments, the compositions described herein may be formulated as thin films or as a powder. In certain embodiments, the compositions described herein may in a form of a thin film, the film being similar to those known in the art (e.g., thin film such as found in a Listerine product on the market) which includes one or more cannabinoid components, a ginger component(s), and a thin film base, wherein the one or more cannabinoid components and the ginger component(s), individually or together, are present in the thin film composition in an effective amount to treat, reduce, and/or prevent nausea and/or vomiting.

In certain embodiments, the compositions described herein may be formulated as suitable for administration via inhalation or nebulization. In certain embodiments, the compositions described herein may be formulated as an aerosol. In certain embodiments, the compositions described herein may be formulated as suitable for administration via smoking or vaping. In certain embodiments, the compositions may be delivered with a suitable delivery device (for example a metered inhaler).

### Chewable Formulation

In certain embodiments, the composition may be a chewable tablet. In certain embodiments, the composition may be a chewable gum. Further embodiments may be described with reference to a chewable composition, however, such description should not be construed as limited to a chewable composition and should be understood as applicable to other oral dosage forms.

For purposes of description, the term “active” is herein defined as the ingredient or ingredients that provide a therapeutic effect. In the present invention active(s) may be provided in either powder or oil form. An active in oil form is defined as a free flowing liquid, semi-solid, or paste that is lipid-based and not water soluble. In the case of a semi-solid or paste, when heated to a maximum temperature of 140 degree F., the material changes to an oil. Active ingredients in oil form can include hemp oil, THC resin, any cannabinoid oil, as well as pharmaceutical actives, botanicals and essential oils.

The chewable composition may include one or more cannabinoid components. Each of the one or more cannabinoid components may be individually present in the oral composition (whether formulated as a chewable composition or as any of the other oral compositions contemplated herein) in a therapeutically effective amount to treat and/or prevent nausea and/or vomiting. In certain embodiments, all the cannabinoid components, together, are present in the oral composition (whether formulated as a chewable composition or as any of the other oral compositions contemplated herein) in a therapeutically effective amount to treat and/or prevent nausea and/or vomiting.

In certain embodiments, the amount of the one or more cannabinoid components, together, in a single dose of the chewable composition ranges from any of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg or about 70 mg to any of about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg.

In certain embodiments, cannabinoid components that may be included in the oral compositions described herein (whether formulated as a chewable composition or as any of the other oral compositions contemplated herein) include cannabidiol, cannabigerol, tetrahydrocannabinol, cannabinol, cannabichromene, or a combination thereof. In one embodiment, the cannabinoid component in the oral compositions described herein (whether formulated as a chewable composition or as any of the other oral compositions contemplated herein) comprise cannabidiol as the sole active ingredient. In one embodiment, the cannabinoid component in the oral compositions described herein (whether formulated as a chewable composition or as any of the other oral compositions contemplated herein) comprise cannabigerol as the sole active ingredient. In one embodiment, the cannabinoid component in the oral compositions described herein (whether formulated as a chewable composition or as any of the other oral compositions contemplated herein) comprise a combination of cannbidiol and cannabigerol as the active ingredients.

In embodiments where the oral compositions described herein (whether formulated as a chewable composition or as any of the other oral compositions contemplated herein) comprises a combination of cannabidiol and cannabigerol, the weight to weight ratio of cannabidiol to cannabigerol ranges from any of about 15:1, about 14:1, about 13:1, about 12:1, about 10:1, about 9:1, or about 8:1 to any of about 7:1, about 6:1, about 5:1, about 4:1, about 3:1, about 2:1, or about 1:1. In certain embodiments, disclosed is a chewable compositions including a combination of cannabidiol and cannabigerol where the weight to weight ratio of cannabidiol to cannabigerol ranges from any of about 15:1, about 14:1, about 13:1, about 12:1, about 10:1, about 9:1, or about 8:1 to any of about 7:1, about 6:1, about 5:1, about 4:1, about 3:1, about 2:1, or about 1:1. In one embodiment, the weight to weight ratio of cannabidiol to cannabigerol ranges from about 15:1 to about 1:1. In one embodiment, the weight to weight ratio of cannabidiol to cannabigerol ranges from about 12:1 to about 2:1. In one embodiment, the weight to weight ratio of cannabidiol to cannabigerol ranges from about 10:1 to about 5:1. In one embodiment, the weight to weight ratio of cannabidiol to cannabigerol is about 8:1.

In certain embodiments, the cannabidiol is present in the oral compositions described herein (whether formulated as a chewable composition or as any of the other oral compositions contemplated herein) in a therapeutically effective amount to treat and/or prevent nausea and/or vomiting. In certain embodiments, the amount of cannabidiol in a single dose of the oral compositions described herein (whether formulated as a chewable composition or as any of the other oral compositions contemplated herein) ranges from any of about 10 mg, about 20 mg, about 30 mg, about 40 mg, about 50 mg, about 60 mg, or about 70 mg to any of about 80 mg, about 90 mg, about 100 mg, about 110 mg, about 120 mg, about 130 mg, about 140 mg, or about 150 mg. In certain embodiments, the amount of cannabidiol in a single dose of the chewable compositions described herein ranges from any of about 10 mg, about 20 mg, about 30 mg, about 40 mg, about 50 mg, about 60 mg, or about 70 mg to any of about 80 mg, about 90 mg, about 100 mg, about 110 mg, about 120 mg, about 130 mg, about 140 mg, or about 150 mg.

In one embodiment, the amount of cannabidiol in a single dose of the oral compositions described herein (whether formulated as a chewable composition or as any of the other oral compositions contemplated herein) ranges from about 10 mg to about 150 mg. In one embodiment, the amount of cannabidiol in a single dose of the chewable compositions described herein ranges from about 10 mg to about 150 mg. In one embodiment, the amount of cannabidiol in a single dose of the oral compositions described herein (whether formulated as a chewable composition or as any of the other oral compositions contemplated herein) ranges from about 40 mg to about 120 mg. In one embodiment, the amount of cannabidiol in a single dose of the chewable compositions described herein ranges from about 40 mg to about 120 mg. In one embodiment, the amount of cannabidiol in a single dose of the oral compositions described herein (whether formulated as a chewable composition or as any of the other oral compositions contemplated herein) ranges from about 70 mg to about 90 mg. In one embodiment, the amount of cannabidiol in a single dose of the chewable compositions described herein ranges from about 70 mg to about 90 mg.

In certain embodiments, the cannabigerol is present in the oral compositions described herein (whether formulated as a chewable composition or as any of the other oral compositions contemplated herein) in a therapeutically effective amount to treat and/or prevent nausea and/or vomiting. In certain embodiments, the amount of cannabigerol in a single dose of the oral compositions described herein (whether formulated as a chewable composition or as any of the other oral compositions contemplated herein) ranges from any of about 1 mg, about 2 mg, about 3 mg, about 4 mg, about 5 mg, about 6 mg, or about 7 mg to any of about 8 mg, about 9 mg, about 10 mg, about 11 mg, about 12 mg, about 13 mg, about 14 mg, about 15 mg, about 16 mg, about 17 mg, about 18 mg, about 19 mg, or about 20 mg. In certain embodiments, the amount of cannabigerol in a single dose of the chewable composition ranges from any of about 1 mg, about 2 mg, about 3 mg, about 4 mg, about 5 mg, about 6 mg, or about 7 mg to any of about 8 mg, about 9 mg, about 10 mg, about 11 mg, about 12 mg, about 13 mg, about 14 mg, about 15 mg, about 16 mg, about 17 mg, about 18 mg, about 19 mg, or about 20 mg.

In certain embodiments, the amount of cannabigerol in a single dose of the oral compositions described herein (whether formulated as a chewable composition or as any of the other oral compositions contemplated herein) ranges from any of about 1 mg to about 20 mg. In one embodiment, the amount of cannabigerol in a single dose of the chewable composition ranges from about 1 mg to about 20 mg. In certain embodiments, the amount of cannabigerol in a single dose of the oral compositions described herein (whether formulated as a chewable composition or as any of the other oral compositions contemplated herein) ranges from any of about 4 mg to about 15 mg. In one embodiment, the amount of cannabigerol in a single dose of the chewable composition ranges from about 4 mg to about 15 mg. In certain embodiments, the amount of cannabigerol in a single dose of the oral compositions described herein (whether formulated as a chewable composition or as any of the other oral compositions contemplated herein) ranges from any of about 8 mg to about 12 mg. In one embodiment, the amount of cannabigerol in a single dose of the chewable composition ranges from about 8 mg to about 12 mg.

In certain embodiments, the cannabidiol and the cannabigerol are present together in the oral compositions described herein (whether formulated as a chewable composition or as any of the other oral compositions contemplated herein) in a therapeutically effective amount to treat and/or prevent nausea and/or vomiting. In certain embodiments, the amount of cannabidiol and the cannabigerol together in a single dose of the oral compositions described herein (whether formulated as a chewable composition or as any of the other oral compositions contemplated herein) ranges from any of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg or about 70 mg to any of about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg.

In certain embodiments, the amount of the cannabidiol and the cannabigerol together in a single dose of the chewable composition ranges from any of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg or about 70 mg to any of about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg.

Currently, cannabis and cannabis products such as can be obtained in the United States and other countries in various forms. For example, cannabis can be obtained with a THC (delta-9-tetrahydrocannabinol) level of less than 0.3% by dry weight of material from cannabis sativa or other varieties of cannabis and is defined as “hemp”. However, cannabis sativa and other varieties also exist with a THC content by dry weight of more than 0.3%, and are commonly known as marijuana.

As used herein, “Cannabis” refers to a genus of flowering plants that includes a single species, Cannabis sativa, which is sometimes divided into two additional species, Cannabis indica and Cannabis ruderalis. These three taxa are indigenous to Central Asia, and. South Asia. Cannabis has long been used for fiber (hemp), for seed and seed oils, for medicinal purposes, and as a recreational drug. The Cannabis can include any physical part of the plant material, including, e.g., the leaf, bud, flower, trichome, seed, or combination thereof. Likewise, the Cannabis can include any substance physically derived from Cannabis plant material, such as, e.g., kief and hashish.

As used herein, “leaf” refers to an organ of a vascular plant, as defined in botanical terms, and in particular, in plant morphology. In reference to Cannabis, the first pair of leaves usually have a single leaflet, the number gradually increasing up to a maximum of about thirteen leaflets per leaf (usually seven or nine), depending on variety and growing conditions. At the top of a flowering plant, this number again diminishes to a single leaflet per leaf. The lower leaf pairs usually occur in an opposite leaf arrangement and the upper leaf pairs in an alternate arrangement on the main stem of a mature plant.

As used herein, “bud” refers to a flower-bearing stem or branch of the Cannabis plant, especially a stem or branch bearing a mass of female flowers with associated leaves. The stem or branch bearing the female flowers can be fresh, or can be dried. The pistils of the female Cannabis flower are surrounded by a mass of trichome-rich petals and leaves, and can contain higher concentrations of cannabinoids than do the plant leaves or stems. A bud, e.g., a mass of female flowers and associated leaves, usually covered with trichomes, can be further processed mechanically, i.e., “trimming” or “cleaning” the stern bearing the female flowers by removal of larger leaves and stem material. Buds, and cleaned buds, can be used as a Cannabis plant material in practice of a method of the invention.

As used herein, “trichome” refers to a fine outgrowth or appendage on plants and certain protists. They are of diverse structure and function. Examples are hairs, glandular hairs, scales, and papillae. In reference to Cannabis, the trichome is a glandular trichome that occurs most abundantly on the floral calyxes and bracts of female plants.

As used herein, “seed” refers to an embryonic plant enclosed in a protective outer covering called the seed coat, usually with some stored food. It is a characteristic of spermatophytes (gymnosperm and angiosperm plants) and the product of the ripened ovule which occurs after fertilization and some growth within the mother plant. The formation of the seed completes the process of reproduction in seed plants (started with the development of flowers and pollination), with the embryo developed from the zygote and the seed coat from the integuments of the ovule.

As used herein, “Cannabis sativa L.” or “Cannabis sativa” refers to an annual herbaceous plant in the Cannabis genus, a species of the Cannabaceae family.

As used herein, “cannabinoid” refers to a class of diverse chemical compounds that act on cannabinoid receptors on cells that repress neurotransmitter release in the brain. These receptor proteins include the endocannabinoids (produced naturally in the body by humans and animals), the phytocannabinoids (found in Cannabis and some other plants), and synthetic cannabinoids (manufactured chemically). The most notable cannabinoid is the phytocannabinoid Δ9-tetrahydrocannabinol (THC), the primary psychoactive compound of Cannabis. Cannabidiol is another major constituent of the plant, representing up to 40% in extracts of the plant resin. There are at least 85 different cannabinoids isolated from Cannabis, exhibiting varied effects.

In certain embodiments, the cannabinoid component (which may be derived from hemp, defined as the plant Cannabis sativa L. and any part of that plant, including the seeds thereof and all derivatives, extracts, cannabinoids, isomers, acids, salts, and salts of isomers thereof) in any of the oral compositions described herein comprises less than about 3 wt %, less than about 2 wt %, less than about 1 wt %, less than about 0.5 wt %, less than 0.3 wt %, less than 0.2 wt %, less than 0.1 wt %, or 0 wt %, tetrahydrocannabinol (THC), the main psychoactive component of cannabis that alters the brain function and induces changes in perception or mood of a user, based on total weight of the cannabinoid component.

In certain embodiments, the cannabinoid component in the chewable composition described herein comprises less than about 3 wt %, less than about 2 wt %, less than about 1 wt %, less than about 0.5 wt %, less than 0.3 wt %, less than 0.2 wt %, less than 0.1 wt %, or 0 wt %, tetrahydrocannabinol (THC), the main psychoactive component of cannabis that alters the brain function and induces changes in perception or mood of a user, based on total weight of the cannabinoid component.

In certain embodiments, the cannabinoid component in any of the oral composition described herein include from any of about 2 wt %, about 3 wt %, about 4 wt %, about 5 wt %, about 6 wt %, about 7 wt %, about 8 wt %, about 9 wt %, or about 10 wt % to any of about 15 wt %, about 20 wt % about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, or about 100 wt %, THC, based on total weight of the cannabinoid component.

In certain embodiments, the cannabinoid component in any of the oral composition described herein includes from about 2 wt % to about 10 wt %, THC, based on total weight of the cannabinoid component. In certain embodiments, the cannabinoid component in any of the oral composition described herein includes from about 10 wt % to about 20 wt %, THC, based on total weight of the cannabinoid component. In certain embodiments, the cannabinoid component in any of the oral composition described herein includes from about 20 wt % to about 30 wt %, THC, based on total weight of the cannabinoid component. In certain embodiments, the cannabinoid component in any of the oral composition described herein includes from about 30 wt % to about 40 wt %, THC, based on total weight of the cannabinoid component. In certain embodiments, the cannabinoid component in any of the oral composition described herein includes from about 40 wt % to about 50 wt %, THC, based on total weight of the cannabinoid component. In certain embodiments, the cannabinoid component in any of the oral composition described herein includes from about 50 wt % to about 60 wt %, THC, based on total weight of the cannabinoid component. In certain embodiments, the cannabinoid component in any of the oral composition described herein includes from about 60 wt % to about 70 wt %, THC, based on total weight of the cannabinoid component. In certain embodiments, the cannabinoid component in any of the oral composition described herein includes from about 70 wt % to about 80 wt %, THC, based on total weight of the cannabinoid component. In certain embodiments, the cannabinoid component in any of the oral composition described herein includes from about 80 wt % to about 90 wt %, THC, based on total weight of the cannabinoid component. In certain embodiments, the cannabinoid component in any of the oral composition described herein includes from about 90 wt % to about 100 wt %, THC, based on total weight of the cannabinoid component.

In certain embodiments, the cannabinoid component in chewable composition described herein include from any of about 2 wt %, about 3 wt %, about 4 wt %, about 5 wt %, about 6 wt %, about 7 wt %, about 8 wt %, about 9 wt %, or about 10 wt % to any of about 15 wt %, about 20 wt % about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, or about 100 wt %, THC, based on total weight of the cannabinoid component.

In certain embodiments, the cannabinoid component in the chewable composition described herein includes from about 2 wt % to about 10 wt %, THC, based on total weight of the cannabinoid component. In certain embodiments, the cannabinoid component in the chewable composition described herein includes from about 10 wt % to about 20 wt %, THC, based on total weight of the cannabinoid component. In certain embodiments, the cannabinoid component in the chewable composition described herein includes from about 20 wt % to about 30 wt %, THC, based on total weight of the cannabinoid component. In certain embodiments, the cannabinoid component in the chewable composition described herein includes from about 30 wt % to about 40 wt %, THC, based on total weight of the cannabinoid component. In certain embodiments, the cannabinoid component in the chewable composition described herein includes from about 40 wt % to about 50 wt %, THC, based on total weight of the cannabinoid component. In certain embodiments, the cannabinoid component in the chewable composition described herein includes from about 50 wt % to about 60 wt %, THC, based on total weight of the cannabinoid component. In certain embodiments, the cannabinoid component in the chewable composition described herein includes from about 60 wt % to about 70 wt %, THC, based on total weight of the cannabinoid component. In certain embodiments, the cannabinoid component in the chewable composition described herein includes from about 70 wt % to about 80 wt %, THC, based on total weight of the cannabinoid component. In certain embodiments, the cannabinoid component in the chewable composition described herein includes from about 80 wt % to about 90 wt %, THC, based on total weight of the cannabinoid component. In certain embodiments, the cannabinoid component in the chewable composition described herein includes from about 90 wt % to about 100 wt %, THC, based on total weight of the cannabinoid component.

In certain embodiments, the chewable composition or any other oral composition further includes a ginger component(s). In certain embodiment, the ginger component(s) is independently present in the chewable composition or in any of the other oral compositions contemplated herein in a therapeutically effective amount to treat and/or prevent nausea and/or vomiting. In certain embodiments, the ginger component(s) (e.g., one or more gingerols, such as, without limitations, 6-gingerol, 8-gingerol, 10-gingerol, 12-gingerol) is the sole active ingredient in the chewable composition or in any of the other oral compositions contemplated herein.

In certain embodiments, the amount of the ginger component(s) in a single dose of the chewable composition or in a single dose of any of the other oral compositions contemplated herein ranges from any of about 1 mg, about 2 mg, about 3 mg, about 4 mg, about 5 mg, about 6 mg, or about 7 mg to any of about 8 mg, about 9 mg, about 10 mg, about 11 mg, about 12 mg, about 13 mg, about 14 mg, about 15 mg, about 16 mg, about 17 mg, about 18 mg, about 19 mg, or about 20 mg. In one embodiment, the amount of ginger component(s) in a single dose of the chewable composition or in a single dose of any of the other oral compositions contemplated herein ranges from about 1 mg to about 20 mg. In one embodiment, the amount of ginger component(s) in a single dose of the chewable composition or in a single dose of any of the other oral compositions contemplated herein ranges from about 4 mg to about 15 mg. In one embodiment, the amount of ginger component(s) in a single dose of the chewable composition or in a single dose of any of the other oral compositions contemplated herein ranges from about 8 mg to about 12 mg.

In certain embodiments, the ginger component(s) together with the one or more cannabinoid components are present in the oral pharmaceutical compositions described herein in a therapeutically effective amount to treat and/or prevent nausea and/or vomiting. In certain embodiments, the ginger component(s) (e.g., gingerol) together with one or more of the cannabinoid components (e.g., cannabidiol and/or cannabigerol) are the active ingredients in the chewable pharmaceutical composition or in any of the other oral compositions contemplated herein.

The weight to weight ratio of the one or more cannabinoid components to the ginger component(s) in the chewable composition or in any of the other oral compositions contemplated herein ranges from any of about 20:1, about 19:1, about 18:1, about 17:1, about 16:1, about 15:1, about 14:1, about 13:1, about 12:1, about 10:1, about 9:1, or about 8:1 to any of about 7:1, about 6:1, about 5:1, about 4:1, about 3:1, about 2:1, or about 1:1.

In one embodiment, the weight to weight ratio of the one or more cannabinoid components to ginger component(s) in the oral pharmaceutical composition described herein ranges from about 15:1 to about 1:1. In one embodiment, the weight to weight ratio of the one or more cannabinoid components to ginger component(s) in the oral pharmaceutical composition described herein ranges from about 12:1 to about 2:1. In one embodiment, the weight to weight ratio of the one or more cannabinoid components to ginger component(s) in the oral pharmaceutical composition described herein ranges from about 10:1 to about 5:1. In one embodiment, the weight to weight ratio of the one or more cannabinoid components to ginger component(s) in the oral pharmaceutical composition described herein is about 9:1.

In certain embodiments, the weight to weight ratios contemplated herein refer to the total weight of all cannabinoid components in the composition relative to the total weight of all ginger components in the composition. For example, in one embodiment, the weight to weight ratios contemplated herein refer to the total weight of the cannabidiol together with the weight of the cannabigerol to the total weight of all of the ginger-derived components in the composition.

In certain embodiments, the weight to weight ratios contemplated herein refer to the weight of a single cannabinoid component in the composition relative to the total weight of all ginger components in the composition. For example, in one embodiment, the weight to weight ratios contemplated herein refer to the weight of the cannabidiol to the total weight of all of the ginger-derived materials in the composition.

In certain embodiments, the weight to weight ratios contemplated herein refer to the total weight of all cannabinoid components in the composition relative to the weight of a single ginger component in the composition. For example, in one embodiment, the weight to weight ratios contemplated herein refer to the total weight of the cannabidiol together with the weight of the cannabigerol to the weight of all of a single gingerol type in the composition.

In certain embodiments, the weight to weight ratios contemplated herein refer to the weight of a single cannabinoid component in the composition relative to the weight of a single ginger component in the composition. For example, in one embodiment, the weight to weight ratios contemplated herein refer to the weight of the cannabidiol to the weight of all of a single gingerol type in the composition.

In certain embodiments, the ginger component(s)s that may be included in the chewable composition include a ginger, a ginger root extract, a shogaol, a zingerone, a gingerol (e.g., one or more of 6-gingerol, 8-gingerol, 10-gingerol, 12-gingerol), or a combination thereof. Other ginger derived materials may also be encompassed by the term “ginger component(s)” as used herein and may also be present in the oral compositions contemplated herein. The weight amounts, weight ratios, and weight percentages for the ginger component(s), as contemplated herein, may refer to a single ginger component or to a plurality of ginger components together.

In one embodiment, the ginger component(s) in the chewable composition or in any of the other oral pharmaceutical composition described herein is gingerol (one type or a combination of several gingerol types). In such embodiments, the gingerol (one type or a combination of several gingerol types) amount in a single dose of the chewable composition or in any of the other oral pharmaceutical composition described herein may range from any of about 1 mg, about 2 mg, about 3 mg, about 4 mg, about 5 mg, about 6 mg, or about 7 mg to any of about 8 mg, about 9 mg, about 10 mg, about 11 mg, about 12 mg, about 13 mg, about 14 mg, about 15 mg, about 16 mg, about 17 mg, about 18 mg, about 19 mg, or about 20 mg. In one embodiment, the amount of gingerol (one type or a combination of several gingerol types) in a single dose of the chewable composition or in any of the other oral pharmaceutical composition described herein ranges from about 1 mg to about 20 mg. In one embodiment, the amount of gingerol (one type or a combination of several gingerol types) in a single dose of the chewable composition or in any of the other oral pharmaceutical composition described herein ranges from about 4 mg to about 15 mg. In one embodiment, the amount of gingerol (one type or a combination of several gingerol types) in a single dose of the chewable composition or in any of the other oral pharmaceutical composition described herein ranges from about 8 mg to about 12 mg.

In certain embodiments, the weight to weight ratio of the cannabidiol and/or cannabigerol (alone or in combination) to the gingerol (one type or a combination of several gingerols) in the chewable composition or in any of the other oral pharmaceutical composition described herein ranges from any of about 20:1, about 19:1, about 18:1, about 17:1, about 16:1, about 15:1, about 14:1, about 13:1, about 12:1, about 10:1, about 9:1, or about 8:1 to any of about 7:1, about 6:1, about 5:1, about 4:1, about 3:1, about 2:1, or about 1:1. In one embodiment, the weight to weight ratio of the cannabidiol and/or cannabigerol (alone or in combination) to gingerol (one type or a combination of several gingerol types) ranges from about 20:1 to about 1:1. In one embodiment, the weight to weight ratio of the cannabidiol and/or cannabigerol (alone or in combination) to gingerol (one type or a combination of several gingerol types) ranges from about 15:1 to about 2:1. In one embodiment, the weight to weight ratio of the cannabidiol and/or cannabigerol (alone or in combination) to gingerol (one type or a combination of several gingerol types) ranges from about 12:1 to about 5:1. In one embodiment, the weight to weight ratio of the cannabidiol and/or cannabigerol (alone or in combination) to gingerol (one type or a combination of several gingerol types) ranges from about 10:1 to about 7:1. In one embodiment, the weight to weight ratio of the cannabidiol and/or cannabigerol (alone or in combination) to gingerol (one type or a combination of several gingerol types) is about 9:1. In one embodiment, the weight to weight ratio of the cannabidiol and/or cannabigerol (alone or in combination) to gingerol (one type or a combination of several gingerol types) is about 8:1.

In certain embodiments, the active ingredient in the chewable composition or in any of the other oral pharmaceutical composition described herein (whether it is one or more of the cannabinoid component with or without the ginger component(s)) is present at a concentration ranging from any of about 0.1 wt %, about 0.2wt %, about 0.3 wt %, about 0.4 wt %, about 0.5 wt %, or about 0.6 wt % to any of about 0.7 wt %, about 0.8 wt %, about 0.9 wt %, about 1 wt %, about 1.1 wt %, about 1.2 wt %, about 1.3 wt %, about 1.4 wt %, or about 1.5 wt %, or any single value or sub-range therein, based on the total weight of the chewable composition or of the other oral pharmaceutical composition described herein.

In certain embodiments, any of the chewable compositions described herein further include a chewable base, e.g. a gum base. A “gum base” refers to a chewable rubber or plastic, which provides the structural integrity of the chewable composition. It may comprise either natural materials (such as the plant resin chicle) or synthetic materials (such as, for example, paraffin wax and related polymers). The gum base may further comprise a combination of elastomers for elasticity, resins to act as binders and softeners, plasticizers to render the elastomer soft to ensure thorough blending of the gum base, waxes, fats, emulsifiers, fillers contributing to the overall texture, antioxidants to prevent oxidation of the gum base, flavors, and combinations thereof as desirable to modify the texture of the chewable composition. In certain embodiments, any of the other oral pharmaceutical compositions described herein may also include pharmaceutically acceptable excipients that may overlap, at least in part, with excipients used in the gum base for the chewable composition.

In certain embodiments, the chewable composition includes a gum base at a concentration ranging from any of about 15 wt %, about 16 wt %, about 17 wt %, about 18 wt %, about 19 wt %, about 20 wt %, about 21 wt %, about 22 wt %, about 23 wt %, about 24 wt %, or about 25 wt % to any of about 26 wt %, about 27 wt %, about 28 wt %, about 29 wt %, about 30 wt %, about 31 wt %, about 32 wt %, about 33 wt %, about 34 wt %, or about 35 wt %, or any single value or sub-range therein, based on the total weight of the chewable composition. In one embodiment, the chewable composition includes the gum base at a concentration ranging from about 20 wt % to about 30 wt %, based on the total weight of the chewable composition.

In certain embodiments, any of the chewable compositions described herein further include additives, such as, without limitations, softeners, plasticizers, glycerin, flavorings, coloring agents, sweeteners, and a fixing agent.

As used herein, the term “additives” refers to non-structural or flavor-related compounds that are added to the chewable composition to impart other desirable properties. These may include various vitamins for imparting healthful effects to the user. In certain embodiments, gingerol may be an additive.

The chewable composition of the present disclosure may also have a softener or plasticizer, such as lecithin, hydrogenated vegetable oils, glycerol ester, lanolin, methyl ester, pentaerythritol ester, rice bran wax, stearic acid, sodium potassium stearates, and the like.

As used herein, the terms “sweeteners” and “flavors” refer to compounds added in order to improve the taste of the chewable composition. It is contemplated that various common sweeteners, such as xylitol and stevia, may be used to enhance the taste of the chewable composition. Other sweeteners used may include: sucrose, dextrose, fructose, glucose or corn syrup, erythritol, isomalt, maltitol, mannitol, xylitol, sorbitol, lactitol, aspartame, acesulfame-K, saccharine, sucralose, neohesperidine, dihydrichalcone. Other flavors used may be natural or synthetic and may be peppermint, spearmint, or a sour acid, such as citric, tartaric, malic, lactic, adipic, and fumaric.

In one embodiment, the sweeteners in the chewable composition include a sugar alcohol or a blend of sugar alcohols that encompasses one or more of sorbitol, isomalt, xylitol, maltitol, mannitol, erythritol, or a combination thereof. The chewable composition includes the sugar alcohol or blend of sugar alcohols at a concentration ranging from any of about 25 wt %, about 30 wt %, about 35 wt %, about 40 wt %, about 45 wt %, about 50 wt %, or about 55 wt % to any of about 60 wt %, about 65 wt %, about 70 wt %, about 75 wt %, or about 80 wt %, or any single value or sub-range therein, based on the total weight of the chewable composition. In one embodiment, the chewable composition includes the sugar alcohol or blend of sugar alcohols at a concentration ranging from about 35 wt % to about 70 wt %, based on the total weight of the chewable composition.

In certain embodiments, additional flavoring in liquid and/or in powder form may be incorporated in the chewable composition described herein. The chewable composition includes the flavoring at a concentration ranging from any of about 5 wt %, about 6 wt %, about 7 wt %, about 8 wt %, or about 9 wt % to any of about 10 wt %, about 11 wt %, about 12 wt %, about 13 wt %, about 14 wt %, or about 15 wt %, or any single value or sub-range therein, based on the total weight of the chewable composition. In one embodiment, the chewable composition includes the flavorings at a concentration ranging from about 9 wt % to about 11 wt %, based on the total weight of the chewable composition.

In certain embodiments, additional intensive sweeteners may be incorporated in the chewable composition described herein. The chewable composition includes the additional intensive sweeteners at a concentration ranging from any of 0%, about 0.1 wt %, about 0.2 wt %, about 0.3 wt %, or about 0.4 wt % to any of about 0.5 wt %, about 0.6 wt %, about 0.7 wt %, about 0.8 wt %, about 0.9 wt %, or about 1 wt %, or any single value or sub-range therein, based on the total weight of the chewable composition. In one embodiment, the chewable composition includes the intensive sweeteners at a concentration ranging from about 0.2 wt % to about 0.6 wt %, based on the total weight of the chewable composition.

As used herein, the term “fixing agent” refers to a polyol that allows the chewable composition to have a hard outer coating. The polyol may be Sorbitol, Maltitol/Isomalt, Mannitol, Starch, and the like.

In certain embodiments, the chewable composition may further include tableting lubricants and/or powder flow agents at a concentration ranging from any of about 1 wt %, about 1.5 wt %, about 2 wt %, about 2.5 wt %, or about 3wt % to any of about 3.5 wt %, about 4 wt %, about 4.5 wt %, about 5 wt %, about 5.5 wt %, about 6 wt %, about 7 wt %, about 8 wt %, about 9 wt %, or about 10 wt %, or any single value or sub-range therein, based on the total weight of the chewable composition.

In certain embodiments, various concentrations, amounts, and weight ratios described herein with respect to a chewable composition may be suitable for any of the other oral pharmaceutical composition contemplated herein.

### Pharmaceutically Acceptable Excipients

In certain embodiments, the compositions described herein may include one or more pharmaceutically acceptable excipients to arrive at a given dosage form having one or more of a target release profile, target stability, target manufacturing process, target in-vitro properties (e.g., dissolution/disintegration), target pharmacokinetic/pharmacodynamics properties, target dosing regimen, and the like.

The term “pharmaceutically acceptable excipient or carrier” refers to any inert ingredient in a composition that may act, for example, to stabilize the active ingredient. A pharmaceutically acceptable excipient can include, but is not limited to, carbohydrates, antioxidants, chelating agents, low-molecular weight proteins, high-molecular weight polymers, gel-forming agents or other stabilizers and additives. Other examples of a pharmaceutically acceptable carrier include wetting agents, emulsifying agents, surfactant and/or dispersing agents, alkalizing agents, coloring agents, synthetic dies, fillers, diluents, mineral oxides, or preservatives, which are particularly useful for preventing the growth or action of microorganisms. Various preservatives are well known and include, for example, phenol and ascorbic acid. Examples of carriers, stabilizers or adjuvants can be found in Remington's Pharmaceutical Sciences, Mack Publishing Company, Philadelphia, Pa., 17th ed. (1985).

In certain embodiments, exemplary pharmaceutically acceptable excipients that may be utilized include, without limitations, acrylics, cellulose derivatives, polysaccharides, monosaccharides, gums, natural or synthetic polymers (e.g., polyalkylene oxides (e.g., polymethylene oxides, polyethylene oxides, polypropylene oxides) polyethylenes, polypropylenes, polyvinyl chlorides, polycarbonates, polystyrenes, polyacrylates, polycaprolactone, polymethacrylates copolymers thereof, and mixtures thereof), liposomes, disintegrants (e.g., polyvinylpyrrolidone, sodium starch glycolate, crosscarmellose sodium, or a mixture thereof), glidants, lubricants, absorption enhancers, surfactants, binders, softeners, plasticizers (e.g., lecithin, hydrogenated vegetable oils, glycerol ester, lanolin, methyl ester, pentaerythritol ester, rice bran wax, stearic acid, sodium potassium stearates, and the like), waxes, fats, emulsifiers, fillers, antioxidants, flavors, colorants, diluents, processing aids (e.g., granulating aids), sweeteners such as those described above with respect to the chewable composition, fixing agents (e.g., polyols such as, without limitations, sorbitol, maltitol/isomalt, mannitol, starch, and the like), pH-adjusting agents, viscosity adjusting agents, solubility increasing or descreasing agents, osmotic agents, solvents, or a combination thereof.

In certain embodiments, suitable exemplary pharmaceutically acceptable excipients may include, without limitations, polyvinylpyrrolidone, natural and synthetic gums, polyvinyl alcohol, corn starch, hydrophilic and hydrophobic materials such as sustained release polymers, acrylic resins, protein-derived materials, waxes, shellacs, and solid or semi-solid oils such as hydrogenated castor oil and hydrogenated vegetable oil. More specifically, the controlled release materials can be, e.g., alkylcelluloses such as ethylcellulose, acrylic and methacrylic acid polymers and copolymers (e.g., acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid) (anhydride), methyl methacrylate, polymethacrylate, poly(methyl methacrylate), poly(methyl methacrylate) copolymer, polyacrylamide, aminoalkyl methacrylate copolymer, poly(methacrylic acid anhydride), glycidyl methacrylate copolymers, and mixtures of any of the foregoing), and cellulose ethers, such as hydroxyalkylcelluloses (e.g., hydroxypropylmethylcellulose) and carboxyalkylcelluloses. Waxes include, e.g., natural and synthetic waxes, fatty acids, fatty alcohols, and mixtures of the same (e.g., beeswax, carnauba wax, stearic acid and stearyl alcohol).

In certain embodiments, various gelling agents can be employed including, for example and without limitation, sugars or sugar derived alcohols, such as mannitol, sorbitol, and the like, starch and starch derivatives, cellulose derivatives (such as microcrystalline cellulose, sodium caboxymethyl cellulose, methylcellulose, ethyl cellulose, hydroxyethyl cellulose, hydroxypropyl cellulose, hydroxypropyl methylcellulose, cellulose esters, cellulose diesters, cellulose triesters, cellulose ethers, cellulose ester-ethers, cellulose acylates, cellulose diacylates, cellulose triacylates, cellulose acetates, cellulose diacetates, cellulose triacetates, cellulose acetate propionates, cellulose acetate butyrates, cellulose acetate succinate, cellulose acetate phthalate, hydroxypropyl methyl cellulose phthalate, hydroxy propyl methyl cellulose acetate succinate (hypermellose acetate succinate), and mixtures thereof), attapulgites, bentonites, dextrins, alginates, algenic acid salts such as sodium alginate and potassium alginate, casein, stearic acid, shellac, carrageenan, gum tragacanth, gum acacia, gum arabic, pullulan gum, dextrin, gellan gum, agar gum, tara gum, karaya, guar gum, welan gum, rhamsan gum, locust bean gum, xanthan gum, pectin, gelatin, kaolin, lecithin, magnesium aluminum silicate, the carbomers and carbopols, polyvinylpyrrolidone, polyethylene glycol, polyethylene oxide, polyvinyl alcohol, silicon dioxide, surfactants, mixed surfactant/wetting agent systems, emulsifiers, other polymeric materials, and mixtures thereof.

In certain embodiments, various hydrophilic excipients may be included in the oral pharmaceutical compositions described herein. Exemplary hydrophilic excipients include, without limitations, water, low molecular weight polyols, such as, polyethylene glycol, polypropylene glycol, or a combination thereof. Examples of other suitable hydrophilic carriers include, without limitations, polyoxyethylene derivatives of a sorbitan ester, such as sorbitan monolaurate (Polysorbate 20), Polysorbate 80, Polysorbate 60, polyoxyethylene 20 sorbitan trioleate (Polysorbate 85), acetic acid, formic acid, other hydrophilic surfactants and mixtures thereof. Exemplary low molecular weight polyols include, without limitations, those having a number average molecular weight of from any of about 200 Dalton, about 400 Dalton, about 600 Dalton, about 800 Dalton, or about 1000 Dalton to any of about 2000 Dalton, about 3000 Dalton, about 4000 Dalton, about 5000 Dalton, about 6000 Da, or about 7000 Da, or any sub-range or single value therein (for instance, polyethylene glycol 400, polyethylene glycol 600, or the like).

In certain embodiments, various plasticizers may be included in the oral pharmaceutical compositions described herein. Suitable plasticizers may include, but not be limited to, sugar alcohol plasticizer such as triacetin, isomalt, maltitol, xylitol, erythritol, adonitol, dulcitol, pentaerythritol, or mannitol; or polyol plasticizer such as diglycerin, ethylene glycol, diethylene glycol, triethyleneglycol, tetraethylene glycol, dipropylene glycol, a polyethylene glycol up to 10,000 MW, neopentyl glycol, propylene glycol, 1,3-propanediol, 2-methyl-1,3-propanediol, trimethylolpropane, a polyether polyol, ethanol amines; and mixtures thereof. Other exemplary plasticizers may also include, without limitations, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, citrate ester-type plasticizers, and triacetin. Such plasticizers may include 1,2-butylene glycol, 2,3-butylene glycol, styrene glycol, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutyl sebacate, acetyltributylcitrate, triethyl citrate, glyceryl monostearate, polysorbate 80, acetyl triethyl citrate, tributyl citrate and allyl glycolate, and mixtures thereof.

In certain embodiments, a suitable plasticizer is selected from the group consisting of phosphate esters; phthalate esters; amides; mineral oils; fatty acids and esters; fatty alcohols, vegetable oils and hydrogenated vegetable oils including acetylated hydrogenated cottonseed glyceride and acetylated hydrogenated soybean oil glycerides; acetyl tributyl citrate, acetyl triethyl citrate, Castor oil, diacetylated monoglycerides, dipropylene glycol salicylate glycerin, glyceryl cocoate, mono- and di-acetylated monoglycerides, nitrobenzene, carbon disulfide, fl-naphtyl salicylate, phthalyl glycolate, diocyl phthalate; sorbitol, sorbitol glyceryl tricitrate; sucrose octaacetate; a-tocopheryl polyethylene glycol succinate, phosphate esters; phthalate esters; amides; mineral oils; fatty acids and esters; fatty alcohols; and vegetable oils, fatty alcohols including cetostearyl alcohol, cetyl alcohol, stearyl alcohol, oleyl alcohol and myristyl alcohol; methyl abietate, acetyl tributyl citrate, acetyl triethyl citrate, diisooctyl adipate, amyl oleate, butyl ricinoleate, benzyl benzoate, butyl and glycol esters of fatty acids, butyl diglycol carbonate, butyl oleate, butyl stearate, di(beta-methoxyethyl) adipate, dibutyl sebacate, dibutyl tartrate, diisobutyl adipate, dihexyl adipate, triethylene glycol di(beta-ethyl butyrate), polyethylene glycol di(2-ethyl hexoate), diethylene glycol monolaurate, monomeric polyethylene ester, hydrogenated methyl ester of rosin, methoxyethyl oleate, butoxyethyl stearate, butyl phthalyl butyl glycolate, glycerol tributyrate, triethylene glycol dipelargonate, beta-(p-tert-amyl phenoxy)ethanol, beta(p-tert-butytphenoxy)ethanol, beta-(p-tert-butytphenoxyethyl)acetate, bis(beta-p-tert-buthylphenoxydiethyl)ether, camphor, Cumar W-1, Cumar MH-1, Cumar V-1, diamyl phthalate, (diamylphenoxy) ethanol, diphenyl oxide, technical hydroabietyl alcohol, beckolin, benzene hexahydrochlonde, Clorafin 40, Piccolastic A-5, Piccalastic A-25, Flexol B-400, Glycerol alfa-methyl alfa-phenyl ether, chlorinated naphthalene, HB-40, monoamylphthalate. Nevillac 10 o-nitrodiphenyl and Paracril 26.

Exemplary suitable coloring agents for the oral pharmaceutical compositions described herein may include, but not be limited to, colors such as e.g., white, black, yellow, blue, green, pink, red, orange, violet, indigo, and brown. In specific embodiments, the color of the dosage form can indicate the contents (e.g., one or more active ingredients) contained therein.

Exemplary suitable flavoring agents for the for the oral pharmaceutical compositions described herein may include, but not be limited to, “flavor extract” obtained by extracting a part of a raw material, e.g., animal or plant material, often by using a solvent such as ethanol or water; natural essences obtained by extracting essential oils from the blossoms, fruit, roots, etc., or from the whole plants.

Additional exemplary flavoring agents for the oral pharmaceutical compositions described herein may include, but not be limited to, breath freshening compounds like menthol, spearmint, and cinnamon, coffee beans, other flavors or fragrances such as fruit flavors (e.g., cherry, orange, grape, etc.), especially those used for oral hygiene, as well as actives used in dental and oral cleansing such as quaternary ammonium bases. The effect of flavors may be enhanced using flavor enhancers like tartaric acid, citric acid, vanillin, or the like.

Exemplary sweetening agents for the for the oral pharmaceutical compositions described herein may include, but not be limited to, one or more artificial sweeteners, one or more natural sweeteners, or a combination thereof. Artificial sweeteners include, e.g., acesulfame and its various salts such as the potassium salt (available as Sunett®), alitame, aspartame (available as NutraSweet® and Equal®), salt of aspartame-acesulfame (available as Twinsweet®), neohesperidin dihydrochalcone, naringin dihydrochalcone, dihydrochalcone compounds, neotame, sodium cyclamate, saccharin and its various salts such as the sodium salt (available as Sweet'N Low®), stevia, chloro derivatives of sucrose such as sucralose (available as Kaltame® and Splenda®), and mogrosides. Natural sweeteners include, e.g., glucose, dextrose, invert sugar, fructose, sucrose, glycyrrhizin; monoammonium glycyrrhizinate (sold under the trade name MagnaSweet®); Stevia rebaudiana (Stevioside), natural intensive sweeteners, such as Lo Han Kuo, polyols such as sorbitol, mannitol, xylitol, erythritol, and the like.

In certain embodiments, suitable excipients include, for example, diluents such as dicalcium phosphate, calcium sulfate, lactose or sucrose or other disaccharides, cellulose, cellulose derivatives, kaolin, mannitol, dry starch, glucose or other monosaccharides, dextrin or other polysaccharides, sorbitol, inositol or mixtures thereof; binders such as acacia, sodium alginate, starch, gelatin, saccharides (including glucose, sucrose, dextrose and lactose), molasses, extract of Irish moss, panwar gum, ghatti gum, mucilage of isapol husk, carboxymethylcellulose, methylcellulose, veegum, larch arabolactan, polyethylene glycols, ethylcellulose, water, alcohols, waxes, polyvinylpyrrolidone such as, e.g., PVP K90 (may be used to improve mixing of the polymer with the other ingredients) or mixtures thereof; lubricants such as talc, magnesium stearate, calcium stearate, staeric acid, hydrogenated vegetable oils, sodium benzoate, sodium chloride, leucine, carbowax 4000, magnesium lauryl sulfate, colloidal silicon dioxide and mixtures thereof, disintegrants such as starches, clays, cellulose derivatives including crosscarmellose, gums, aligns, various combinations of hydrogencarbonates with weak acids (e.g. sodium hydrogencarbonate/tartaric acid or citric acid) crosprovidone, sodium starch glycolate, agar, cation exchange resins, citrus pulp, veegum HV, natural sponge, bentonite or mixtures thereof; volatile solvents such as alcohols, including aqueous alcohols, petroleum benzine, acetone, ether or mixtures thereof; plasticizers such as sorbitol and glycerine; and others such as cocoa butter, polyethylene glycols or polyethylene oxides, e.g. with a molecular weight of about 1,000-500,000 daltons, typically about 1,000-100,000 daltons, more typically 1,000-50,000 daltons, especially about 1,000-10,000 daltons, in particular about 1,500-5,000 daltons, and mixtures thereof, hydrogenated vegetable oils, glycerinated gelatin or mixtures thereof.

In certain embodiments, suitable antioxidants may include BHA, BHT, t-butyl hydroquinone, calcium ascorbate, gallic acid, hydroquinone, maltol, octyl gallate, sodium bisulfate, sodium metabisulfite, tocopherol and derivates thereof, citric acid, tartaric acid, and ascorbic acid. Other antioxidants include trivalent phosphorous like e.g phosphite, phenolic antioxidants, hydroxylamines, lactones such as substituted benzofuranones. Hindered phenols, thiosynergists and/or hindered amines are useful for the long-term stability for polymers, whereas the following antioxidants are suitable for use also in situation where the active substance is subject to oxidation: acids (ascorbic acid, erythorbic acid, etidronic acid, gallic acid, hypophosphorous acid, nordihydroguairetic acid, propionic acid etc.), phenols (e.g. BHA, BHT, t-butyl hydroquinone, dodecyl gallate, octyl gallate, 1,3,5-trihydroxybenzene), organic and inorganic salts (calcium ascorbate, sodium ascorbate, sodium bisulphite, sodium metabisulfite, sodium sulfite, potassium bisulphite, potassium metabisulphite), esters (calcium ascorbate, dilauryl thiodipropionate, dimyristyl thiodipropionate, distearyl thiodipropionate), pyranon (maltol), and vitamin E (tocopherol, D-α-tocopherol, DL-α-tocopherol, tocopherol acetate, d-α-tocopheryl acetate, dl-α-tocopheryl acetate. However, other anti-oxidative agents known in the art may be used according to the present invention.

In certain embodiments, suitable antioxidants may include, without limitations, sterically hindered phenols, aryl amines, thioureas, thiocarbamates, phosphites, thioether esters, and combinations of the foregoing. Other suitable examples of antioxidants include, but are not limited to, alkylated monophenols, including but not limited to, 2,6-di-tert-butyl-4-methylphenol, 2-tert-butyl-4,6-di-methylphenol, 2,6-di-tert-butyl-4-ethylphenol, 2,6-di-tert-butyl-4-n-butylphenol, 2,6-di-tert-butyl-4-isobutylphenol, 2,6-dicyclopentyl-4-methylphenol, 2-(α-methylcyclohexyl)-4,6-dimethylphenol, 2,6-dioctadecyl-4-methylphenol, 2,4,6-tricyclohexylphenol, 2,6-di-tert-butyl-4-methoxymethylphenol, nonylphenols which are linear or branched in the side chains, for example, 2,6-di-nonyl-4-methylphenol, 2,4-dimethyl-6-(1′-methylundec-1′-yl)phenol, 2,4-dimethyl-6-(1′-methylheptadec-1′-yl)phenol, 2,4-dimethyl-6-(1′-methyltridec-1-yl)phenol and mixtures thereof, alkylthiomethylphenols, including but not limited to, 2,4-dioctylthiornethyl-6-tert-hutylphenol, 2,4-dioctylthiomethyl-6-methylphenol, 2,4-dioetylthiomethyl-6-ethylphenol, 2,6-di-dodecylthiomethyl-4-nonylphenol, hydroquinones and alkylated hydroquinones, including but not limited to, 2,6-di-tert-hutyl-4-methoxyphenol, 2,5-di-tert-butylhydroquinone, 2,5-di-tort-amylhydroquinone, 2,6-diphenyl-4-octadecyloxyphenol, 2,6-di-tert-butylhydroquinone, 2,5-di-tert-butyl-4-hydroxyanisole, 3,5-di-tert-butyl-4-hydroxyanisole, 3,5-di-tert-butyl-4-hydroxyphenyl stearate, bis(3,5-di-tert-butyl-4-hydroxyphenyl) adipate, tocopherols, including but not limited to, α-tocopherol, β-tocopherol, γ-tocopherol, δ-tocopherol and mixtures thereof (vitamin E), hydroxylated thiodiphenyl ethers, including but not limited to, 2,2′-thiobis(6-tort-butyl-4-methylphenol), 2,2′-thiobis(4-oetylphenol), 4,4′-thiobis(6-tert-butyl-3-methylphenol), 4,4′-thiobis(6-tert-butyl -2-methylphenol), 4,4′-thiobis(3,6-di-sec-amylphenol), 4,4′-bis(2,6-dimethyl-4-hydroxyphenyl)-disulfide, alkylidenebisphenols, including but not limited to, 2,2′-methylenebis(6-tert-butyl-4-methylphenol), 2,2′-methylenebis(6-tert-butyl-4-ethylphenol), 2,2′-methylenebis[4-methyl-6-(α-methylcyclohexyl)-phenol], 2,2′-methylenebis(4-methyl-6-cyclohexylphenol), 2,2′-methylenebis(6-nonyl-4-methylphenol), 2,2′-methylenebis(4,6-di-tert-butylphenol), 2,2′-ethylidenebis(4,6-di-tert-butylphenol), 2,2′-ethylidenebis(6-tert-butyl-4-isobutylphenol), 2,2′-methylenebis [6-(α-methylbenzyl)-4-nonylphenol], 2,2′-methylenebis[6-(α,α-dimethylbenzyl)-4-nonylphenol], 4,4′-methylenebis(2,6-di-tert-butylphenol), 4,4′-methylenebis(6-tert-butyl-2-methylphenol), 1,1-bis(5-tert-butyl-4-hydroxy-2-methylphenyl)butane, 2,6-bis(3-test-butyl-5-methyl-2-hydroxybenzyl)-4-methylphenol, 1,1,3-tris(5-tert-butyl-4-hydroxy-2-methylphenyl)butane, 1,1-bis(5-tert-butyl-4-hydroxy-2-methyl-phenyl)-3-n-dodecylmercaptobutane, ethylene glycol bis[3,3-bis(3′-tert-butyl-4′-hydroxyphenyl)butyrate], bis(3-tert-butyl-4-hydroxy-5-methyl-phenyl)dicyclopentadiene, bis[2-(3′-tert-butyl-2′-hydroxy-5-methylbenzyl)-6-tert-butyl-4-methylphenyl]terephthalate, 1,1-bis-(3,5-dimethyl-2-hydroxyphenyl)butane, 2,2-bis(3,5-di-tert-butyl-4-hydroxyphenyl)propane, 2,2-bis(5-tert-butyl-4-hydroxy-2-methylphenyl)-4-n-dodecylmercaptobutane, 1,5,5-tetra-(5-tert-butyl-4-hydroxy-2-methylphenyl)pentane, O-, N- and S-benzyl compounds, including but not limited to, 3,5,3′,5′-tetra-tert-butyl.-4,4′-dihydroxydibenzyl ether, octadecyl-4-hydroxy-3,5-dimethylbenzylmercaptoacetate, tridecyl-4-hydroxy-3,5-di-tert-butylbenzylmercaptoacetate, tris(3,5-di-tert-butyl-4-hydroxybenzyl)amine, bis(4-tert-butyl-3-hydroxy-2,6-dimethylbenzyl)dithioterephthalate, bis(3,5-di-tert-butyl-4-hydroxybenzyl)sulfide, isooctyl-3,5-di-tert-butyl-4-hydroxybenzylmercaptoacetate, hydroxybenzylated malonates, including but not limited to, dioctadecyl-2,2-bis(3,5-di-tert-butyl-2-hydroxybenzyl)malonate, di-octadecyl-2-(3-tert-butyl-4-hydroxy-5-methylbenzyl)malonate, didodecylmercaptoethyl-2,2-bis(3,5-di-tert-butyl-4-hydroxybenzyl)malonate, bis[4-(1,1,3,3-tetramethylbutyl)phenyl]-2,2-bis(3,5-di-tert-butyl-4-hydroxybenzyl)malonate, aromatic hydroxybenzyl compounds, including but not limited to, 1,3,5-tris(3,5-di-tert-butyl-4-hydroxybenzyl)-2,4,6-trimethylbenzene, 1,4-bis(3,5-di-tert-butyl-4-hydroxybenzyl)-2,3,5,6-tetramethylbenzene, 2,4,6-tris(3,5-di-tert-butyl-4-hydroxybenzyl)phenol, triazine compounds, including but not limited to, 2,4-bis(octylmercapto)-6-(3,5-di-tert-butyl-4-hydroxyanilino)-1,3,5-triazine, 2-octylmercapto-4,6-bis(3,5-di-tert-butyl-4-hydroxyanilino)-1,3,5-triazine, 2-octylmercapto-4,6-bis(3,5-di-tert-butyl-4-hydroxyphenoxy)-1,3,5-triazine, 2,4,6-tris-(3,5-di-tert-butyl-4-hydroxyphenoxy)-1,2,3-triazine, 1,3,5-tris(3,5-di-tert-butyl-4-hydroxybenzyl)isocyanurate, 1,3,5-tris(4-tert-butyl-3-hydroxy-2,6-dimethylbenzyl)isocyanurate, 2,4,6-tris-(3,5-di-tert-butyl-4-hydroxyphenylethyl)-1,3,5-triazine, 1,3,5-tris(3,5-di-tert-butyl-4-hydroxy-phenylpropionyl)-hexahydro-1,3,5-triazine, 1,3,5-tris(3,5-dicyclohexyl-4-hydroxybenzyl)iso-cyanurate, benzylphosphonates, including but not limited to, dimethyl-2,5-di-tert-butyl-4-hydroxybenzylphosphonate, diethyl-3,5-di-tert-butyl-4-hydroxybenzylphosphonate, dioctadecyl3,5-di-tent-butyl-4-hydroxybenzylphosphonate, dioctadecyl-5-tert-butyl-4-hydroxy-3-methylbenzylphosphonate, the calcium salt of the monoethyl ester of 3,5-di-tert-butyl-4-hydroxybenzylphosphonic acid, acylaminophenols, including but not limited to, 4-hydroxylauranilide, 4-hydroxystearanilide, octyl N-(3,5-di-tert-butyl-4-hydroxyphenyl)carbamate, esters of β-(3,5-di-tert-butyl-4-hydroxyphenyl)propionic acid with mono- or polyhydric alcohols, e.g. with methanol, ethanol n-octanol, i-octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane, esters of β-(5-tert-butyl-4-hydroxy-3-methylphenyl)propionic acid with mono- or polyhydric alcohols, e,g. with methanol, ethanol, n-octanol, i-octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis-(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2] octane; 3,9-bis[2-{3-(3-tert-butyl-4-hydroxy-5-methylphenyl)propionyloxy}-1,1-dimethylethyl]-2,4,8,10- -tetraoxaspiro[5.5]-undecane, esters of 6-(3,5-dicyclohexyl-4-hydroxyphenyl)propionic acid with mono- or polyhydric alcohols, e.g. with methanol, ethanol, octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2,2]octane, esters of 3,5-di-tert-butyl-4-hydroxyphenyl acetic acid with mono- or polyhydric alcohols, e.g. with methanol, ethanol, octanol, octadecanol, 1,6-hexanediol, 1,9-nonanediol, ethylene glycol, 1,2-propanediol, neopentyl glycal, thiodiethyl.ene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane, amides of 6-(3,5-di-tert-butyl-4-hydroxyphenyl)propionic acid e.g. N,N′-bis(3,5-di-tert-butylA-hydroxyphenylpropionyl)hexamethylenediamide, N,N′-bis(3,5-di-tert-butyl-4-hydroxyphenylpropionyl)trimethylenediamide, N,N′-bis(3,5-di-tert-butyl-4-hydroxyphenylpropionyphydrazide, N,N′-bis[2-(3-[3,5-di-tert-butyl-4-hydroxyphenyl]propionyloxy)ethyl]oxamide (Naugard®XL-1, supplied by Uniroyal), ascorbic acid (vitamin C), aminic antioxidants, including but not limited to, N,N′-di-isopropyl-p-phenylenediamine, N,N′-di-sec-butyl-p-phenylenediamine, N,N′-bis(1,4-dimethylpentyl)-p-phenylenediamine, N,N′-bis(1-ethyl-3-methylpentyl)-p-phenylenediamine, N,N′-bis(1-methylheptyl)-p-phenylenediamine, N,N′-dicyclohexyl-p-phenylenediamine, N,N′-diphenyl-p-phenylenediamine, N,N′-bis(2-naphthyl)-p-phenylenediamine, N-isopropyl-N′-phenyl-p-phenylenediamine, N-(1,3-dimethylbutyl)-N′-phenyl-p-phenylenediamine, N-(1-methylheptyl)-N′-phenyl-p-phenylenediamine, N-cyclohexyl-N′-phenyl-p-phenylenediamine, 4-(p-toluenesulfamoyl)diphenylamine, N,N′-dimethyl-N,N′-di-sec-butyl-p-phenylenediamine, diphenylamine, N-allyldiphenylamine, 4-isopropoxydiphenylamine, N-phenyl-1-naphthylamine, N-(4-tert-octylphenyl)-1-naphthylamine, N-phenyl-2-naphthylamine, octylated diphenyl amine, including but not limited to, p,p′-di-tert-octyldiphenylamine, 4-n-butylaminophenol, 4-butyrylaminophenol, 4-nonanoylaminophenol, 4-dodecanoylaminophenol, 4-octadecanoylaminophenol, bis(4-methoxyphenyl)amine 2,6-di-tert-butyl-4-dimethylaminomethylphenol, 2,4′-diaminodiphenylmethane, 4,4′-diaminodiphenylmethane, N,N,N′,N′-tetramethyl-4,4′-diaminodiphenylmethane, 1,2-bis[(2-methylphenyl)amino]ethane, 1,2-bis(phenylamino)propane, (o-tolyl)biguanide, bis[4-(1′,3′-dimethylbutyl)phenyl]amine, tert-octylated N-phenyl-1-naphthylamine, a mixture of mono- and dialkylated tert-butyl/tert-octyldiphenylamines, a mixture of mono- and dialkylated nonyldiphenylamines, a mixture of mono- and dialkylated dodecyldiphenylamines, a mixture of mono- and dialkylated isopropyl/isohexyldiphenylamines, a mixture of mono- and dialkylated teak-butyldiphenylamines, 2,3-dihydro-3,3-dimethyl-4H-1,4-b enzothiazine, phenothiazine, a mixture of mono- and dialkylated tert-butyl/tert-octylphenothiazines, a mixture of mono- and dialkylated tert-octyl-phenothiazines, N-allylphenothiazine, N,N,N′,N′-tetraphenyl-1,4-diaminobut-2-ene, and combinations of the foregoing.

In certain embodiments, the oral pharmaceutical compositions may include alkalizing agent(s), such as, without limitations, magnesium oxide, ammonium hydroxide, sodium hydroxide, sodium carbonate, sodium citrate, trisodium phosphate and/or disodium phosphate.

In certain embodiments, the oral pharmaceutical compositions may include lubricant(s)/release agent(s) such as, but not limited to, fatty acids and their salts, fatty alcohols, fatty esters, fatty amines, fatty amine acetates and fatty amides. Other suitable lubricants may include, but not be limited to, glyceryl behenate (Compritol™ 888), metallic stearates (e.g., magnesium, calcium and sodium stearates), stearic acid, hydrogenated vegetable oils (e.g., Sterotex™), talc, waxes such as beeswax and carnauba wax, silica, fumed silica, colloidal silica, calcium stearate, long chain fatty alcohols, boric acid, sodium benzoate and sodium acetate, sodium chloride, DL-Leucine, polyethylene glycols (e.g., Carbowax™ 4000 and Carbowax™ 6000), sodium oleate, sodium benzoate, sodium acetate, sodium lauryl sulfate, sodium stearyl fumarate (Pruv™), magnesium lauryl sulfate, stearic acid, stearyl alcohol, mineral oil, paraffin, micro crystalline cellulose, glycerin, propylene glycol and combinations thereof.

In certain embodiments, the oral pharmaceutical compositions may include diluents such as, but not limited to, lactose USP, lactose USP (anhydrous), lactose USP (spray dried), starch USP, directly compressible starch, mannitol USP, sorbitol, dextrose monohydrate, microcrystalline cellulose NF, dibasic calcium phosphate dihydrate NF, sucrose-based diluents, confectioner's sugar, monobasic calcium sulfate monohydrate, calcium sulfate dihydrate NF, calcium lactate trihydrate granular NF, dextrates NF (e.g., Emdex™), dextrose (e.g., Cerelose™), inositol, hydrolyzed cereal solids such as the Maltrons™ and Mor-Rex™, amylose, powdered cellulose (e.g., Elcema™), calcium carbonate, glycine, bentonite, polyvinylpyrrolidone, and the like.

In certain embodiments, the oral pharmaceutical compositions may include oils and fats such as, but not be limited to, almond oil, argan oil, avocado oil, canola oil, cashew oil, castor oil, cocoa butter, coconut oil, colza oil, corn oil, cottonseed oil, grape seed oil, hazelnut oil, hemp oil, hydroxylated lecithin, lecithin, linseed oil, macadamia oil, mango butter, manila oil, mongongo nut oil, olive oil, palm kernel oil, palm oil, peanut oil, pecan oil, perilla oil, pine nut oil, pistachio oil, poppy seed oil, pumpkin seed oil, rice bran oil, safflower oil, sesame oil, shea butter, soybean oil, sunflower oil, walnut oil, and watermelon seed oil. Other oil and fats that may be in the fill of the PVA shell may include, but not be limited to, fish oil (omega-3), crill oil, animal or vegetable fats, e.g., in their hydrogenated form, mono-, di-, and tri-glycerides with C12-, C14-, C16-, C18-, C20- and C22-fatty acids.

In certain embodiments, the oral pharmaceutical compositions may include pH modifiers such as, but not be limited to, hydrochloric acid, potassium hydroxide, sodium hydroxide, ammonium hydroxide, sulfuric acid, phosphoric acid, and nitric acid.

In certain embodiments, the oral pharmaceutical compositions may include other exemplary excipients such as, but not be limited to, vegetable proteins such as sunflower protein, soybean proteins, cotton seed proteins, peanut proteins, grape seed proteins, whey proteins, whey protein isolates, blood proteins, egg proteins, acrylated proteins, water-soluble polysaccharides such as alginates, carrageenans, guar gum, agar-agar, xanthan gum, gellan gum, gum arabic and related gums (gum ghatti, gum karaya, gum tragancanth), pectin, water-soluble derivatives of cellulose: alkylcelluloses hydroxyalkylcelluloses and hydroxyalkylalkylcelluloses, such as methylcelulose, hydroxymethylcellulose, hydroxyethylcellulose, hydroxypropylcellulose, hydroxyethylmethylcellulose, hydroxypropylmethylcellulose, hydroxybutylmethylcellulose, cellulose esters and hydroxyalkylcellulose esters such as cellulose acetate phthalate (CAP), hydroxypropylmethylcellulose (HPMC); carboxyalkylcelluloses, carboxyalkylalkylcelluloses, carboxyalkylcellulose esters such as carboxymethylcellulose and their alkali metal salts; water-soluble synthetic polymers such as polyacrylic acids, polyacrylamides, and polyacrylic acid esters, polymethacrylic acids, polymethacrylamides, and polymethacrylic acid esters, polyvinylacetates, polyvinylalcohols, polyvinylacetatephthalates (PVAP), polyvinylpyrrolidone (PVP), PVY/vinyl acetate copolymer, and polycrotonic acids; also suitable are phthalated gelatin, gelatin succinate, crosslinked gelatin, shellac, water-soluble chemical derivatives of starch, cationically modified acrylates and methacrylates possessing, for example, a tertiary or quaternary amino group, such as the diethylaminoethyl group, which may be quaternized if desired; and other similar polymers; inorganic fillers, such as the oxides of magnesium aluminum, silicon, titanium, etc.

In certain embodiments, the oral pharmaceutical compositions may include other pharmaceutically acceptable excipients such as, without limitations, a hydrophobic material, including, but not limited to, digestible, long chain (C₈-C₅₀, especially C₁₂-C₄₀), substituted or unsubstituted hydrocarbons, such as natural or synthetic waxes (such as beeswax, glycowax, castor wax and carnauba wax), fatty alcohols (such as lauryl, myristyl, stearyl, cetyl or preferably cetostearyl alcohol), fatty acids, including, but not limited to, mono-diglyceride of medium chain fatty acids (such as caprylic, capric, caproic, lauric, oleic, linoleic), medium chain triglycerides, fatty acid esters, fatty acid glycerides (mono-, di-, and tri-glycerides), hydrogenated fats, hydrocarbons, normal waxes, stearic acid, stearyl alcohol and hydrophobic and hydrophilic materials having hydrocarbon backbones.

In certain embodiments, the oral pharmaceutical compositions may include other pharmaceutically acceptable excipients such as, without limitations, polyvinyl alcohols, polyvinyl pyrrolidone, polyalkylene oxides, polyacrylic acid, cellulose, cellulose ethers, cellulose esters, cellulose amides, polyvinyl acetates, polycarboxylic acids and salts, acetic acid, caprylic acid, oleic acid, polyaminoacids or peptides, polyamides, polyacrylamide, copolymers of maleic/acrylic acids, polysaccharides including starch and gelatin, natural gums such as xanthan, and carrageenans. For example, polymers can be selected from polyacrylates and water-soluble acrylate copolymers, methylcellulose, carboxymethylcellulose sodium, dextrin, ethylcellulose, hydroxyethyl cellulose, hydroxypropyl methylcellulose, maltodextrin, polymethacrylates, and combinations thereof, or selected from polyvinyl alcohols, polyvinyl alcohol copolymers and hydroxypropyl methyl cellulose (HPMC), methacrylic acid/methyl methacrylate, methacrylic acid/ethyl acrylate copolymers, methacrylic acid/methyl acrylate/methyl methacrylate copolymers, shellac, hydroxypropyl methylcellulose phthalate, hydroxyl propyl methyl cellulose acetate succinate, hydroxypropyl methyl cellulose trimellitate, cellulose acetate phthalates, polyvinyl acetate phthalates, PEG-35 castor oil, caprylocaproyl polyoxyl-8 glycerides, glyceryl distearate, and combinations thereof.

In certain embodiments, the oral pharmaceutical compositions may include high HLB surfactants such as, without limitations, polysorbate 80-polyoxyethylene (20) sorbitan monooleate, polyoxyl 40 hydrogenated castor oil, polyoxyl 35 castor oil, caprylocaproyl macrogol glycerides, and combinations thereof.

In certain embodiments, the oral pharmaceutical compositions may include fillers such as, without limitations, lactose, microcrystalline cellulose, and combinations thereof.

Other pharmaceutically acceptable excipients may also be utilized as recognized by those skilled in the art.

In certain embodiments, pharmaceutically acceptable excipients may be included in the oral compositions described herein in a concentration ranging from any of about 5 wt %, about 10 wt %, about 15 wt %, about 20 wt %, about 25 wt %, about 30 wt %, about 35 wt %, about 40 wt %, about 45 wt %, or about 50 wt % to any of about 55 wt %, about 60 wt %, about 65 wt %, about 70 wt %, about 75 wt %, about 80 wt %, about 85 wt %, about 90 wt %, about 95 wt %, or about 99 wt %, or any sub-range or single value therein based on the total weight of the oral pharmaceutical composition.

### Release Profile

In certain embodiments, any of the oral compositions described herein may be formulated to have a target release profile, such as, without limitations, controlled release, immediate release, enteric release, delayed release, targeted release in a location within the gastrointestinal tract (e.g., in the stomach, the duodenum, colonic delivery and the like), first order release profile, zero order release profile, pulsatile release, or any combination thereof. In certain embodiments, any of the oral compositions described herein may be coated with a suitable coating (e.g., to attain a certain release profile) and/or with a cosmetic coating.

In certain embodiments, the oral compositions described herein have an immediate release profile. The term “immediate release,” as used herein refers to an oral composition releasing at least about 85%, at least about 90% or at least about 95% of the active agent within 15 minutes, within 30 minutes, within 45 minutes, or within 60 minutes as measured by in-vitro dissolution in a USP Apparatus 2 (basket) or in a USP Apparatus 2 (paddle) or in a USP Apparatus 3 (reciprocating cylinder) or in a USP Apparatus 4 (flow-through cell system) suitable conditions as recognized by those skilled in the art in accordance with industry guidelines provided by a given regulatory authority.

In certain embodiments, the oral compositions described herein have a controlled release profile. The term “controlled release,” as used herein refers to an oral composition releasing the active agent over a period of time, e.g., to provide a once daily or twice daily dosage form.

In certain embodiments, the oral compositions described herein have a delayed release profile with a targeted release in a certain location within the gastrointestinal tract, for instance, through pH dependent dissolution or disintegration. In one embodiment, the oral compositions described herein may preferentially release the active agent in the stomach. In one embodiment, the oral compositions described herein may preferentially release the active agent in the colon. In one embodiment, the oral compositions described herein may preferentially release the active agent in the duodenum.

In certain embodiments, the oral compositions described herein have a zero order release rate, such that the release rate of the active agent is constant over a period of time.

In certain embodiments, the oral compositions described herein have a first order release rate, such that the release rate of the active agent is proportional to the concentration of the active agent in the oral composition.

### Method of Treatment

In certain embodiments, the instant disclosure relates to a method for treating subjects in need thereof with any of the chewable compositions described herein. The subject in need thereof may be a cancer subject that is either experiencing nausea and/or vomiting or is being treated prophylactically against nausea and/or vomiting that may be developed as a result of the cancer treatment regime, the cancer itself, other drugs, and other nausea and/or vomiting causes as understood by one skilled in the art. In certain embodiments, the subject in need thereof may be a subject experiencing physiological nausea and/or vomiting that may be triggered by peripheral factors such as ingestion of toxins (e.g., alcohol/drug intoxication), disturbance of the vestibular system, or peritoneal inflammation and bowel obstruction. In certain embodiments, the subject in need thereof may be a subject experiencing nausea and/or vomiting related to disorders of delayed gastric emptying as, for example, diabetes and idiopathic gastroparesis. In certain embodiments, the subject in need thereof may be a subject experiencing nausea and/or vomiting that is psychogenic and may be triggered by anxiety, threatening situation, a situation that is regarded as distasteful by the subject, or by the subject's hostility (such as a temper tantrum). In certain embodiments, the subject in need thereof may be a subject experiencing the nausea and/or vomiting that may be induced by cytotoxic chemotherapy and/or radiotherapy. In certain embodiments, the subject in need thereof may be a subject experiencing nausea and/or vomiting that may be post-operative and may be attributed to the anesthetic agents and/or the analgesic agents that are administered to the subject. In certain embodiments, the subject in need thereof may be a subject that is experiencing nausea and/or vomiting related to motion sickness. In certain embodiments, the subject in need thereof may be a subject experiencing nausea and/or vomiting that is pregnancy related.

In certain embodiments, administering includes causing the subject to chew the chewable composition. In certain embodiments, administering includes chewing the chewable composition by the subject (e.g., cancer subject). The chewable composition may be administered once a week, once every three days, every other day, once a day, twice a day, three times a day, four times a day, or on an as needed basis.

In certain embodiments, administering includes causing the subject to apply the composition or to place the composition in the oral cavity. In certain embodiments, the composition that is applied or placed in the oral cavity is to remain undisturbed while the composition disintegrates and/or dissolves.

In certain embodiments, the composition may remain undisturbed in the oral cavity of the subject for a duration sufficient to deliver a therapeutically effective amount of the active ingredient to alleviate, minimize, treat, and/or prevent the occurrence of nausea and/or vomiting. In certain embodiments, the chewable composition may be chewed by the subject for a duration sufficient to deliver a therapeutically effective amount of the active ingredient to alleviate, minimize, treat, and/or prevent the occurrence of nausea and/or vomiting. The chewing duration and/or the duration for holding the composition in the oral cavity of the subject undisturbed may range from any of about 1 minute, about 5 minutes, about 10 minutes, about 15 minutes, about 20 minutes, or about 25 minutes to any of about 30 minutes, about 35 minutes, about 40 minutes, about 45 minutes, about 50 minutes, about 55 minutes, or about 60 minutes, or any single duration value or duration sub-range therein.

In certain embodiments, the compositions described herein may be administered concurrently, simultaneously, or sequentially along with other drugs as part of the treatment regimen for a given condition. In certain embodiments, the chewable composition may be administered to a subject concurrently, simultaneously, or sequentially along with other drugs as part of a treatment regimen for a given condition. In one embodiment, the compositions described herein may be administered to a subject in need thereof concurrently, simultaneously, or sequentially along with drugs prescribed as part of a cancer treatment regimen.

The term “concurrently” as used herein means that a dose of one agent (e.g., the chewable composition) is administered prior to the end of the dosing interval of another agent (e.g., cancer treatment active agent).

The term “simultaneously” as used herein means that a dose of one agent (e.g., the chewable composition) is administered approximately at the same time as another agent (e.g., cancer treatment active agent) regardless of whether the agents are administered separately via the same or different routes of administration or in a single pharmaceutical composition or dosage form.

The term “sequentially” as used herein means that a dose of one agent (e.g., the chewable composition) is administered first and thereafter a dose of another agent is administered second (e.g., cancer treatment active agent). The subsequent administration of the second agent may be inside or outside the dosing interval of the first agent.

### Method of Manufacturing

In certain embodiments, the instant disclosure relates to a method for manufacturing any of the chewable compositions or any of the other oral pharmaceutical compositions described herein (whether chewable or non-chewable). Generally, the method includes combining one or more cannabinoid components with one or more ginger components and a chewable base (e.g., gum base) to form a chewable composition. In certain embodiments, generally, the method includes combining one or more cannabinoid components with one or more ginger components and one or more pharmaceutically acceptable excipient to form any of the other oral compositions described herein. A variety of methods may be used to combine these components, some of which are described in further detail below. The below description should not be construed as limiting as other suitable methods may also be implemented.

In certain embodiments, the method for manufacturing a chewable composition includes heating the chewable base in an oven to melt the chewable base to an internally measured temperature ranging from any of about 100° F., about 120° F., or about 140° F. to any of about 160° F., 180° F., or about 200° F. In a separate vessel, such as a mixer, the constituents of the chewable composition, such as the one or more cannabinoid components (e.g., cannabidiol and/or cannabigerol and/or gingerol) are combined and mixed. The constituents may be in powder form or in oil form. The melted chewable base is added to the mixer, where the combination is cooled to produce a particulate mixture. The temperature of the gum base exceeds that of the mixer when first introduced, but as mixing continues it cools quickly to room temperature and forms rock-sized granular pieces. These granular pieces are then conditioned for a period of time, which allows the granular pieces to dry slightly and complete the crystallization process. In certain embodiments, the granular pieces are conditioned for at least 6 hours at a temperature not greater than about 75° F. and 60% relative humidity. Next, the pieces are ground into a powder at room temperature, mixed at room temperature with tableting excipients, and tableted in a tablet press. This avoids extreme heating or cooling of the active ingredient(s) and preserves the efficacy.

The prepared chewable composition may subsequently be packaged into a suitable container. Suitable containers include, without limitations, a bottle, a bag, a blister package, wrappers, or any other suitable packaging for a chewable composition. In certain embodiments, the instant disclosure is also directed to a kit that includes a container and any of the pharmaceutical compositions described herein stored within the container.

In certain embodiments, the instant disclosure relates to methods of manufacturing any of the oral compositions described herein. In certain embodiments, the instant disclosure may be directed to methods of manufacturing oral dosage forms formulated for oral ingestion, for mucosal administration such as via the sublingual route, buccal route, gingival route, for the entirety of the oral cavity (entire mouth space), or a combination thereof. In certain embodiments, the instant disclosure may be directed to methods of manufacturing pharmaceutical compositions in a form of a tablet, a capsule, caplets, a lozenge, a troche, a chewable tablet (such as a gum), a syrup, a liquid solution or suspension, an emulsion, a buccal film, a sublingual film, an oral adhesive film, a powder, solid crystals, an orally-disintegrating tablet, a paste, an oral cream, an oral gel, an oral ointment, and so on. Exemplary manufacturing procedures include, without limitations, compression (e.g., to form a compressed tablet and optionally a compression coating), granulation (e.g., wet granulation or dry granulation), tableting, spray drying, freeze drying, extrusion, rotary die encapsulation, blending, milling, mixing, autoclaving, sterilizing, compaction, coating, packaging, combinations thereof, and the like. The skilled artisan would recognize the various manufacturing steps to be taken to arrive at a given dosage form.

### EXAMPLES

The following examples are set forth to assist in understanding the invention and should not be construed as specifically limiting the invention described and claimed herein. Such variations of the invention, including the substitution of any or all equivalents now known or later developed, which would be within the purview of those skilled in the art, and changes in formulation or minor changes in therapeutic design, are to be considered to fall within the scope of the invention incorporated herein.

Table 1 below depicts an exemplary formulation of a chewable composition according to certain embodiments.

TABLE 1
Exemplary Formulation of a Chewable Composition

| | | |
|---|---|---|
| A sugar alcohol or a blend of sugar alcohols | 35.0-70.0 | wt % |
| (e.g., one or more of sorbitol, isomalt, xylitol, | | |
| maltitol, mannitol, or erythritol) | | |
| Gum Base | 20.0-30.0 | wt % |
| Flavoring in liquid and powder form | 9.0-11.0 | wt % |
| Active Ingredients (e.g., one or more of | 0.5-10.0 | wt % |
| cannabidiol, cannabigerol, and gingerol) | | |
| Tableting lubricants and powder flow agents | 1.5-5.0 | wt % |
| Intensive Sweeteners | 0.2-0.6 | wt % |

In the foregoing description, numerous specific details are set forth, such as specific materials, dimensions, processes parameters, etc., to provide a thorough understanding of the present invention. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is simply intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. Reference throughout this specification to “an embodiment”, “certain embodiments”, or “one embodiment” means that a particular feature, structure, or characteristic described in connection with the embodiment is included in at least one embodiment. Thus, the appearances of the phrase “an embodiment”, “certain embodiments”, or “one embodiment” in various places throughout this specification are not necessarily all referring to the same embodiment.

The present invention has been described with reference to specific exemplary embodiments thereof. The specification and drawings are, accordingly, to be regarded in an illustrative rather than a restrictive sense. Various modifications of the invention in addition to those shown and described herein will become apparent to those skilled in the art and are intended to fall within the scope of the appended claims.

1. A chewable composition comprising:
one or more cannabinoid components in an effective amount to treat nausea and/or vomiting;
a ginger component; and
a chewable base.

2. The chewable composition of claim 1, wherein the one or more cannabinoid components comprises cannabidiol, cannabigerol, tetrahydrocannabinol, cannabinol, cannbichromene, or a combination thereof.

3. The chewable composition of claim 2, wherein the one or more cannabinoid components comprises cannabidiol.

4. The chewable composition of claim 3, wherein the one or more cannabinoid components comprises cannabigerol.

5. The chewable composition of claim 4, wherein the w/w ratio of the cannabidiol to the cannabigerol ranges from about 15:1 to about 1:1.

6. The chewable composition of claim 1, wherein the ginger component comprises gingerol.

7. The chewable composition of claim 1, wherein the w/w ratio of the one or more cannabinoid components to the ginger component ranges from about 15:1 to about 5:1, from about 12:1 to about 7:1, from about 10:1 to about 8:1, or about 9:1.

8. The chewable composition of claim 3, wherein the w/w ratio of the cannabidiol to the ginger component ranges from about 20:1 to about 1:1.

9. The chewable composition of claim 1, wherein the chewable base comprises a gum base.

10. A chewable composition comprising:
one or more cannabinoid components;
a ginger component; and
a chewable base;
wherein the one or more cannabinoid components and the ginger component, together, are present in the chewable composition in an effective amount to treat nausea and/or vomiting.

11. The chewable composition of claim 1, wherein the chewable composition comprises less than 0.3 wt % THC (delta-9-tetrahydrocannabinol), based on total weight of the one or more cannabinoid components.

12. A chewable composition comprising:
an effective amount of cannbidiol to treat nausea and/or vomiting;
a cannabigerol;
a ginger component; and
a chewable base.

13. A method for treating nausea and/or vomiting, the method comprising:
administering to a subject in need thereof a chewable composition comprising one or more cannabinoid components and a ginger component.

14. The method of claim 13, wherein the subject is a cancer subject.

15. The method of claim 13, wherein administering comprises chewing the chewable composition.

16. The method of claim 13, wherein the one or more cannabinoid components comprises cannabidiol, cannabigerol, tetrahydrocannabinol, cannabinol, cannbichromene, or a combination thereof.

17. The method of claim 16, wherein the one or more cannabinoid components comprises less than 0.3 wt % THC (delta-9-tetrahydrocannabinol), based on total weight of the one or more cannabinoid components.

18. The method of claim 13, wherein the one or more cannabinoid components comprises cannabidiol.

19. The method of claim 18, wherein the one or more cannabinoid components further comprises cannabigerol.

20. The method of claim 19, wherein the w/w ratio of the cannabidiol to the cannabigerol ranges from about 15:1 to about 1:1.

21. The method of claim 13, wherein the ginger component comprises gingerol.

22. The method of claim 13, wherein the w/w ratio of the one or more cannabinoid components to the ginger component ranges from about 15:1 to about 5:1.

23. The method of claim 18, wherein the w/w ratio of the cannabidiol to the ginger component ranges from about 20:1 to about 1:1.

24. The method of claim 13, wherein the chewable base comprises a gum base.

25. The method of claim 13, wherein the one or more cannabinoid components is present in the chewable composition in an effective amount to treat nausea and/or vomiting.

26. The method of claim 13, wherein the one or more cannabinoid components and the ginger component, together, are present in the chewable composition in an effective amount to treat nausea and/or vomiting.

27. The method of claim 18, wherein the cannabidiol is present in the chewable composition in an effective amount to treat nausea and/or vomiting.

28. The method of claim 18, wherein the cannabidiol and the ginger component, together, are present in the chewable composition in an effective amount to treat nausea and/or vomiting.

29. The method of claim 13, wherein the nausea and/or vomiting is triggered by one or more of an underlying medical condition, motion sickness, pregnancy, psychogenic factors, substance intoxication, or post operation.

30. A method for preparing a chewable composition according to claim 1, the method comprising:
combining the one or more cannabinoid components, the ginger component and the chewable base to form a chewable composition.

31. An oral pharmaceutical composition comprising:
an effective amount of cannbidiol to treat nausea and/or vomiting;
a cannabigerol;
a ginger component; and
a pharmaceutically acceptable excipient.

32. The oral pharmaceutical composition of claim 31, wherein the oral pharmaceutical composition is formulated for oral ingestion, sublingual administration, buccal administration, gingival administration, or a combination thereof.

33. The oral pharmaceutical composition of claim 32, wherein the oral pharmaceutical composition is in a form of a tablet, a capsule, caplets, a lozenge, a troche, a chewable tablet, a gum, a gummy, a syrup, a liquid solution, a suspension, an emulsion, a buccal film, a sublingual film, an oral adhesive film, a powder, solid crystals, an orally-disintegrating tablet, a paste, an oral cream, an oral gel, or an oral ointment.

34. A method for preparing the oral pharmaceutical composition of claim 31, the method comprising combining the cannabidiol, the cannabigerol, and the ginger component with the pharmaceutically acceptable excipient to form the oral pharmaceutical composition.

35. A method for treating nausea and/or vomiting in a subject in need thereof, the method comprising administering, placing, or applying the oral pharmaceutical composition of claim 31 into the oral cavity of the subject.
